SEQUENCE LISTING (32 sequences)

SEQ ID NO: 1 - DNA, 31 bp, Artificial sequence
  Synthetic oligonucleotide
gagagctcaagcttcaaaatgtttctactcc

SEQ ID NO: 2 - DNA, 33 bp, Artificial sequence
  Synthetic oligonucleotide
ggccgcggcaaaacttagattagattgctatgc

SEQ ID NO: 3 - DNA, 52 bp, Artificial sequence
  Synthetic oligonucleotide
gaccgcgggaaataatgtctatggttagtaaaggagaagaaaataacatggc

SEQ ID NO: 4 - DNA, 42 bp, Artificial sequence
  Synthetic oligonucleotide
gggcggccgcttattatttgtatagttcatccatgccaccag

SEQ ID NO: 5 - DNA, 32 bp, Artificial sequence
  Synthetic oligonucleotide
gagcggccgcgagggccgcatcatgtaattag

SEQ ID NO: 6 - DNA, 29 bp, Artificial sequence
  Synthetic oligonucleotide
ggtctagaggccgcaaattaaagccttcg

SEQ ID NO: 7 - DNA, 32 bp, Artificial sequence
  Synthetic oligonucleotide
ggtctagacgccttgagcctggcgaacagttc

SEQ ID NO: 8 - DNA, 62 bp, Artificial sequence
  Synthetic oligonucleotide
agtaaaaaaggagtagaaacattttgaagctatcgatgacaggatgaggatcgtttcgcatg

SEQ ID NO: 9 - DNA, 61 bp, Artificial sequence
  Synthetic oligonucleotide
catgcgaaacgatcctcatcctgtcatcgatagcttcaaatgtttctactccttttttact

SEQ ID NO: 10 - DNA, 41 bp, Artificial sequence
  Synthetic oligonucleotide
ggggatcccaaaacttagattagattgctatgctttctttc

SEQ ID NO: 11 - DNA, 32 bp, Artificial sequence
  Synthetic oligonucleotide
gagagctcatagcttcaaaatgtttctactcc

SEQ ID NO: 12 - DNA, 32 bp, Artificial sequence
  Synthetic oligonucleotide
gggaattctttgtaattaaaacttagattaga

SEQ ID NO: 13 - DNA, 28 bp, Artificial sequence
  Synthetic oligonucleotide
gacctaggaaacaaacaaagctgtcacc

SEQ ID NO: 14 - DNA, 34 bp, Artificial sequence
  Synthetic oligonucleotide
ggctcgagtttttatttttctttttgctgtttcg

SEQ ID NO: 15 - DNA, 37 bp, Artificial sequence
  Synthetic oligonucleotide
aagagctcatagcttcaaaatgtctctactccttttt

SEQ ID NO: 16 - DNA, 40 bp, Artificial sequence
  Synthetic oligonucleotide
aaaggatccaacttagattagattgctatgctttctttcc

SEQ ID NO: 17 - DNA, 35 bp, Artificial sequence
  Synthetic oligonucleotide
aaaggatccaattaatagtgcacatgcaatttcac

SEQ ID NO: 18 - DNA, 31 bp, Artificial sequence
  Synthetic oligonucleotide
aaaacctaggttaaggaagaaacatcatctg

SEQ ID NO: 19 - DNA, 32 bp, Artificial sequence
  Synthetic oligonucleotide
tttgcggccgcccaatctcagaggctgagtct

SEQ ID NO: 20 - DNA, 33 bp, Artificial sequence
  Synthetic oligonucleotide
tttggatcctttgattttcagaaacttgatggc

SEQ ID NO: 21 - DNA, 80 bp, Artificial sequence
  Synthetic oligonucleotide
gtatacgtgattaagcacacaaaggcagcttggagtatgtctgttattaatttcacaggaagattgtactgagagtgcac

SEQ ID NO: 22 - DNA, 80 bp, Artificial sequence
  Synthetic oligonucleotide
ttgcttttcaaaaggcctgcaggcaagtgcacaaacaatacttaaataaatactactcagcgactcactatagggagacc

SEQ ID NO: 23 - DNA, 57 bp, Artificial sequence
  Synthetic oligonucleotide
tccatggtatggatgaattgtacaaataaagcctaggaaacaaacaaagctgtcacc

SEQ ID NO: 24 - DNA, 64 bp, Artificial sequence
  Synthetic oligonucleotide
aagaaattcgcttatttagaagtggcgcgcctctcgagtttttatttttctttttgctgtttcg

SEQ ID NO: 25 - DNA, 30 bp, Artificial sequence
  Synthetic oligonucleotide
ggtcacaaattggaatacaactataactct

SEQ ID NO: 26 - DNA, 23 bp, Artificial sequence
  Synthetic oligonucleotide
cggaattaaccctcactaaaggg

SEQ ID NO: 27 - DNA, 28 bp, Artificial sequence
  Synthetic oligonucleotide
gacctaggaaacaaacaaagctgtcacc

SEQ ID NO: 28 - DNA, 34 bp, Artificial sequence; identical to SEQ ID NO: 14
  Synthetic oligonucleotide

SEQ ID NO: 29 - DNA, 50 bp, Artificial sequence
  Synthetic oligonucleotide
ttctaatacgactcactatagggacctaggaaacaaacaaagctgtcacc

SEQ ID NO: 30 - DNA, 34 bp, Artificial sequence; identical to SEQ ID NO: 14
  Synthetic oligonucleotide

SEQ ID NO: 31 - DNA, 32 bp, Artificial sequence
  Synthetic oligonucleotide
ggtgacagctttgtttgtttcctaggtccccc

SEQ ID NO: 32 - DNA, 17 bp, Artificial sequence
  Synthetic oligonucleotide
gctgtttcgtcctcacg

(19) United States
(12) Patent Application Publication
(10) Pub. No.: US 2012/0184460 A1
(43) Pub. Date: Jul. 19, 2012

(54) HIGHLY EFFICIENT GENE-REGULATORY ELEMENT SCREENING ASSAY AND COMPOSITIONS FOR PERFORMING THE SAME

(76) Inventors: Joe C. Liang, San Gabriel, CA (US); Christina D. Smolke, Stanford, CA (US)

(21) Appl. No.: 13/350,597

(22) Filed: Jan. 13, 2012

Related U.S. Application Data
(60) Provisional application No. 61/432,287, filed on Jan. 13, 2011.

(51) Int. Cl. C40B 30/10; C12M 1/34; G01N 21/64

(52) U.S. Cl. 506/12; 435/29; 435/288.7

(57) ABSTRACT

Methods of evaluating a gene-regulatory element, including libraries of known or candidate gene-regulatory elements, are provided. Aspects of the methods include cytometrically analyzing a cell comprising a gene-regulatory element construct, e.g., a plasmid, having an activity reporter comprising a first signal reporter domain which produces a first signal operatively coupled to a gene-regulatory element of interest; and a noise reporter comprising a second signal reporter which produces a second signal that is distinguishable from the first signal to obtain the first and second signals. The first signal is then normalized with the second signal to obtain a normalized activity signal, which normalized activity signal is then employed to evaluate the gene-regulatory element. Also provided are reagents, systems and kits that find use in practicing methods of the invention.

### CROSS-REFERENCE TO RELATED APPLICATIONS

Pursuant to 35 U.S.C. §119 (e) this application claims priority to the filing date of U.S. Provisional Patent Application Ser. No. 61/432,287 filed Jan. 13, 2011; the disclosure of which application is herein incorporated by reference.

### STATEMENT OF GOVERNMENT SUPPORT

This invention was made with Government support under contract RO1 GM086663 awarded by the National Institutes of Health and contract CBET-0917638 awarded by the National Science Foundation. The Government has certain rights in this invention.

### INTRODUCTION

The ability to develop new biological functions is increasingly important to advancing our ability to engineer biological systems that address challenges faced in health and medicine, environment, and sustainability. One important class of biological functions is encoded in gene-regulatory elements, which allow for the design of sophisticated genetic circuits programming cellular behavior (Win & Smolke, “Higher-order cellular information processing with synthetic RNA devices,” Science (2008) 322: 456-460; Elowitz & Leibler, “A synthetic oscillatory network of transcriptional regulators,” Nature (2000) 403: 335-338; Guet et al., “Combinatorial synthesis of genetic networks,” Science (2002) 296: 1466-1470.) Gene-regulatory elements are composed of one or more components, which encode more basic functions, such as sensing, information transmission, and actuation. Biological components are typically derived from naturally occurring sequences, such as a hammerhead ribozyme (Pley et al., “Three-dimensional structure of a hammerhead ribozyme,” Nature (1994) 372: 68-74), or generated de novo through in vitro evolution strategies, such as an aptamer (Jenison et al., “High-resolution molecular discrimination by RNA,” Science (1994) 263: 1425-1429).

An example of a gene-regulatory element of interest is a modular ligand-responsive ribozyme-based platform that supports efficient tailoring of RNA device function (Win & Smolke, “A modular and extensible RNA-based gene-regulatory platform for engineering cellular function,” Proc, Nat'l Acad. Sci. USA (2007) 104: 14283-14288). The ribozyme device platform specifies physical linkages between three functional RNA components: a sensor encoded by an RNA aptamer, an actuator encoded by a hammerhead ribozyme, and a transmitter encoded by a sequence that undergoes a strand-displacement event. An example of such a control device is illustrated in FIG. 1. Ribozyme-based devices are constructed by modular assembly of three functional RNA components. A sensor (RNA aptamer) is linked to an actuator (hammerhead ribozyme) through a distinct information transmitter sequence (which directs a strand-displacement event and insulates the sensor and actuator components). Ribozyme-based devices are integrated into the 3′ untranslated region (UTR) of the target gene and can adopt at least two functional device conformations, where each conformation is associated with different actuator and sensor activities. In the depicted example, a ribozyme ON device (up-regulation of gene expression in response to increased input ligand concentration) adopts a ribozyme-active conformation associated with an aptamer ligand-unbound state, where ribozyme cleavage results in an unprotected transcript that is subject to rapid degradation by ribonucleases, thereby leading to a decrease in gene expression. The ribozyme-inactive conformation is associated with an aptamer ligand-bound state, such that ligand binding to the aptamer stabilizes the ribozyme-inactive conformation, thereby leading to an increase in gene expression in response to ligand.

Several in vitro selection strategies have been developed to enhance the activities of gene-regulatory elements, but often activities optimized in the in vitro environment may not translate into optimal activities in the cellular environments.

### SUMMARY

Methods of evaluating a gene-regulatory element (including libraries of candidate gene-regulatory elements) are provided. Aspects of the methods include cytometrically analyzing a cell comprising a gene-regulatory element construct, e.g., a plasmid, having an activity reporter comprising a first signal reporter domain which produces a first signal operatively coupled to a gene-regulatory element of interest; and a noise reporter comprising a second signal reporter which produces a second signal that is distinguishable from the first signal to obtain the first and second signals. The first signal is then normalized with the second signal to obtain a normalized activity signal, which normalized activity signal is then employed to evaluate the gene-regulatory element. Also provided are reagents, systems and kits that find use in practicing methods of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Schematic representation of modular assembly and mechanism of an RNA control device based on a ribozyme actuator.

FIG. 2. Schematic representations of various types of screening constructs that differ from each other with respect to the gene-regulatory element.

FIG. 3. A high-efficiency, quantitative cell-based screening strategy for genetic devices based on a two-color screening construct. (A) The two-color screening construct is composed of two independent activity reporters. The device activity reporter measures the gene expression activity associated with the regulatory device from GFP fluorescence, whereas the noise reporter measures the cellular gene expression activity that is independent of the regulatory device from mCherry fluorescence. (B) Single-color (GFP) scatter plots of three ribozyme-based devices that span a wide range of activities, as measured by their mean values, and cellular autofluorescence from a construct containing no fluorescence reporter gene exhibit significant overlap due to noise associated with gene expression. (C) Single-color (GFP) histograms illustrate that isolation of a device with a specific regulatory activity based on a single reporter output is inefficient due to overlapping population distributions. (D) The gene expression activities of individual cells can be normalized by correlating the device and noise reporter outputs from the two-color screening construct. Cell populations harboring the three ribozyme-based devices in (B) can be cleanly resolved on a two-color scatter plot, where each population exhibits a tight linear relationship between the two outputs. (E) The two-color screening strategy is based on the output correlation between the two reporter modules. A library of control devices can be integrated in the two-color screening construct and transformed into the target cell host. The sorting gate is set by the two-color correlation (slope) associated with a control device that exhibits desired activity and applied to the library to specifically isolate a cell population that exhibits similar activity (slope).

FIG. 4. Screening of a sensor library within the device platform demonstrates the high enrichment efficiency of the two-color sorting strategy. (A) A sensor library, sN10, is generated by randomizing 10 nts at key positions within the aptamer component in a previously engineered theophylline-responsive ribozyme-based device, L2b8. (B) The sN10 library is subjected to two sorting rounds. Each round consists of one negative sort in the absence of theophylline (light green gate set by the activity of the parent L2b8 device in the absence of theophylline), followed by one positive sort in the presence of theophylline (dark green gate set by the activity of the parent L2b8 device in the presence of 5 mM theophylline). Percentage of cells collected in the sorting gate is indicated on each plot. (C) After two sorting rounds, ˜80% of the enriched sensor library exhibits a clear population shift in response to theophylline (theo).

FIG. 5. Screening of an actuator library within the device platform results in ribozyme variants that exhibit improved gene-regulatory stringencies and cleavage rates. (A) An actuator library, aN7, is generated by randomizing 7 nts at key positions within the loop I region of the ribozyme actuator in the L2b8 device. (B) The aN7 library is subjected to a single sort to enrich for devices that exhibit lower basal gene expression levels than the parent L2b8 device. The majority (˜99%) of the aN7 library exhibits a greater slope than that of the parent L2b8 device, such that one sort is sufficient to isolate members that exhibit improved regulatory stringency. (C) Ribozyme variants isolated from the aN7 library screen exhibit lower basal activities relative to the parent L2b8 device. Gene-regulatory activities are reported as the geometric mean of the GFP fluorescence of the indicated sample normalized to that of a positive control (sTRSV Contl, a noncleaving sTRSV ribozyme with a scrambled core) that is grown under identical ligand conditions and is set to 100%. Reported values are the mean and standard deviation of at least three independent experiments. (D) The recovered ribozyme variants (L2b8-a1, -a14) exhibit faster cleavage rates than the parent device (L2b8). Cleavage assays were performed at 37° C. with 500 μM MgCl₂, 100 mM NaCl, 50 mM Tris-HCl (pH 7.0). Cleavage rate constants (k) and errors are reported as the mean and standard deviation from at least three independent assays. (E) In vitro cleavage kinetics of the ribozyme variants (L2b8-a1, -a14) and the parent device (L2b8). The projected cleavage kinetics are generated from the single-exponential equation F₁=F₀+(F_{∞}−F₀)×(1−e^{-kt}), setting the fraction cleaved before the start of the reaction (F₀) and at reaction endpoint (F_{∞}) to 0 and 1, respectively, and k to the experimentally determined value for each RNA device.

FIG. 6. Screening of transmitter libraries within the device platform results in transmitter variants that exhibit improved activation ratios. (A) Two transmitter libraries, tN11 and a1-tN11, are generated by randomizing 11 nts within the transmitter components in the L2b8 (wild-type ribozyme actuator) and L2b8-a1 (enhanced ribozyme actuator) devices, respectively. (B) The tN11 library is subjected to one sorting round (negative and positive sort), followed by an additional positive sort to further enrich members of the library that exhibit equal or greater increases in gene-regulatory activities in response to theophylline. The negative (light green) and positive (dark green) sorting gates are set based on the activity of the parent L2b8 device in the absence and presence of 5 mM theophylline, respectively. (C) Transmitter variants isolated from the tN11 library screen exhibit improved activation ratios. Gene-regulatory activities are reported as described in FIG. 5C. Reported values are the mean and standard deviation of at least three independent experiments. The activation ratio (AR) is determined as the ratio of gene expression levels in the presence and absence of theophylline. (D) The a1-tN11 library is subjected to one sorting round to enrich members of the library that exhibit equal or greater increased in gene-regulatory activities in response to theophylline. The negative (light green) and positive (dark green) sorting gates are set based on the activity of the parent L2b8-a1 device in the absence and presence of 5 mM theophylline, respectively. (E) Transmitter variants isolated from the a1-tN11 library screen exhibit improved activation ratios. Gene-regulatory activities are reported as described in FIG. 5C. Reported values are the mean and standard deviation of at least three independent experiments.

FIG. 7. In vitro cleavage kinetics of selected ribozyme-based devices and controls. Cleavage assays were performed at 37° C. with 500 μM MgCl₂, 100 mM NaCl and 50 mM Tris-HCl (pH 7.0) and in the presence of 5 mM theophylline when indicated. (A) Ribozyme-based devices exhibit decreased cleavage rates in the presence of ligand. Cleavage assays were performed as described in FIG. 5D in the absence or presence of 5 mM theophylline. (B) In vitro cleavage kinetics of ribozyme-based devices in the absence and presence of ligand. Projected cleavage kinetics are generated as described in FIG. 5E. Solid lines: 0 mM theophylline; dashed lines: 5 mM theophylline. (C) Correlation analysis of normalized gene expression levels and cleavage rate constants indicates a strong correlation between the in vivo gene-regulatory activity and in vitro cleavage rate; Pearson correlation coefficient (r) of −0.9018. A semi-log line is well-fit (R²=0.94) for cleavage rates less than or equal to 0.16 min⁻¹ (black data points). Devices excluded from this analysis are indicated in red.

FIG. 8. Fate routing in the yeast mating pathway with RNA devices. (A) Signaling through the yeast mating pathway starts with pheromone (α-factor) binding to a transmembrane receptor. Receptor binding is transmitted to the scaffold-bound canonical three-tiered MAPK cascade via a phosphorylation relay. Msg5 antagonizes signaling by dephosphorylating Fus3. Translocation of the phosphorylated MAPK, Fus3, to the nucleus initiates transcription at mating genes. Pathway activation is indicated by increased GFP levels (from a transcriptional fusion to the mating-responsive promoter FUS1p) and cell cycle arrest (observed via halo assays). (B) Schematic representation of fate routing constructs, where ribozyme-based devices and controls are placed downstream of a TEF1 m7p-Msg5 cassette. (C) Gene-regulatory activities of RNA devices and controls utilized to regulate the yeast mating pathway. Gene-regulatory activities are reported as described in FIG. 5C. Reported values are the mean and standard deviation of at least three independent experiments. (D) Stringent control of a negative regulator component in the yeast mating pathway is necessary to regulate pathway activation. Normalized pathway activity is reported as the geometric mean of three biological replicates normalized to FUS1p-yEGFP3 expression in the absence of MSG5 expression, and error bars represent the standard deviation of the replicates. (E) Stringent control of a negative regulatory component in the yeast mating pathway is necessary to route cellular fate. Halo formation is a direct read-out of pheromone-induced cell cycle arrest, where reduced halo formation in the presence of pheromone indicates pathway inhibition. Notably, halo formation is inhibited for the systems implementing L2b8 and sTRSV Contl as controllers in the absence or presence of theophylline. In contrast, the system implementing L2b8-a1 exhibits normal halo formation in the absence of theophylline (comparable to sTRSV) and significantly reduces halo formation in the presence of theophylline. Representative images from three biological replicates are shown.

FIG. 9. Linear correlation between mCherry and GFP fluorescence for L2b1 (A), L2b5 (B), and L2b8 (C) devices within the two-color construct. For each device, 2,000 cells are plotted from a single representative experiment at 0 and 5 mM theophylline (theo). Linear regression and R² values are reported and support a distinct slope for different device regulatory activities.

FIG. 10. Comparison of resolution between single-color and two-color screens. Enriched sN10 library after one sorting cycle and an additional negative sort was analyzed through flow cytometry in the presence and absence of 5 mM theophylline. The positive sorting gate is set by the activity of the parent device in the presence of 5 mM theophylline. In the absence of theophylline, the gate contains cells that are false positives; in the presence of theophylline, the gate contains both true and false positives. (A) The left GFP histogram represents the enriched library in the absence of theophylline, while the right GFP histogram represents the enriched library in the presence of theophylline. For the single-color screen, a hypothetical sorting gate was set based on the percentage of cells isolated from the corresponding two-color screen (4.32%). If a single-color screen were performed, ˜64% (2.77/4.35) of the isolated cells would be false positives. (B) The left two-color scatter plot represents the enriched library in the absence of theophylline, while the right two-color scatter plot represents the enriched library in the presence of theophylline. The two-color screen exhibits high resolving power, such that only ˜6% (0.27/4.32) of the isolated cells were false positives, resulting in a significant improvement in enrichment efficiency over the single-color screen.

FIG. 11. The two-color sorting strategy supports separation of autofluorescent and low expression cell populations. sTRSV (the active ribozyme control) exhibits ˜1.2% normalized GFP fluorescence, relative to a noncleaving ribozyme positive control (sTRSV Contl) with a scrambled core. (A) A cell population harboring the sTRSV construct exhibits low fluorescence levels that overlap significantly with a cell population harboring no fluorescence reporter gene in the single-color histogram. (B) The sTRSV and autofluorescent populations separate based on mCherry expression in the two-color plot, allowing cells exhibiting low GFP expression due to stringent device gene-regulatory activity to be effectively enriched through two-color sorting.

FIG. 12. An actuator sort preserves switching activity in 6 out of 8 characterized devices. Basal activity of the parent device, L2b8, is indicated by a dashed line. Gene-regulatory activities are reported as the geometric mean of the GFP fluorescence of the indicated sample normalized to that of a positive control (sTRSV Contl, a noncleaving sTRSV ribozyme with a scrambled core) that is grown under identical ligand conditions and is set to 100%. Reported values are the mean and standard deviation of at least three independent experiments. The activation ratio (AR), determined as the ratio of gene expression levels in the presence and absence of theophylline (theo), is indicated for each device.

FIG. 13. Point mutation analyses of variant loop I sequences identify consensus sequences of ribozyme variants supporting improved gene-regulatory activities. The impact of point mutations to the recovered loop I sequences on basal activity was measured by flow cytometry. Relative expression levels are reported as the geometric mean of the GFP fluorescence of the indicated sample normalized to that of the L2b8 parent device. Reported values are the mean and standard deviation of at least three independent experiments. Two putative loop I consensus sequences were supported from the sequences recovered from the actuator library screen: a heptaloop (AUYNRRG) and a triloop (NUYGGGN^{l}).

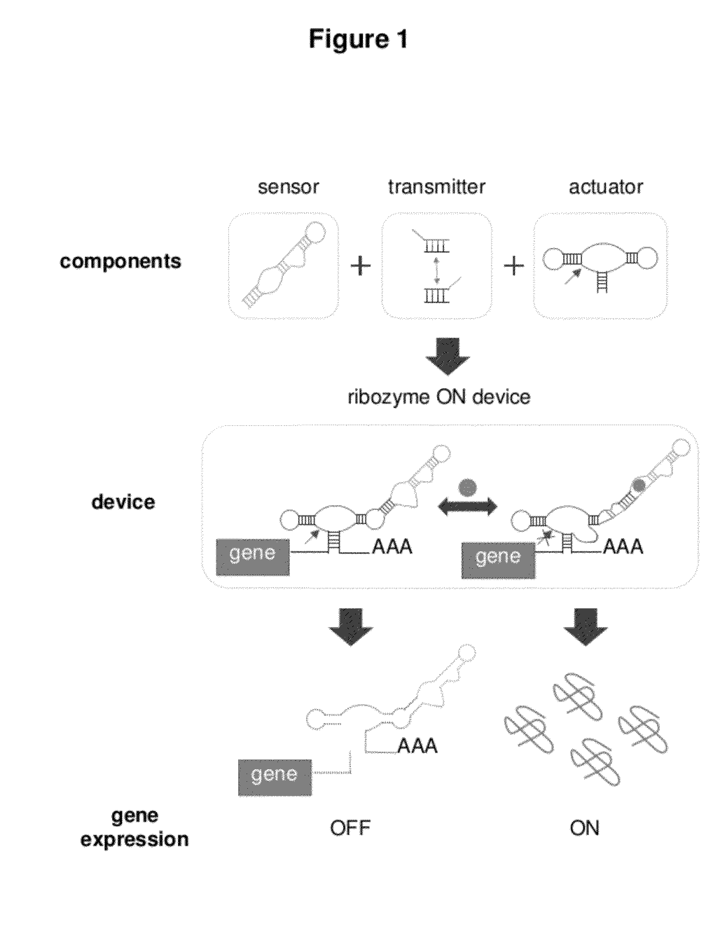

Analysis of the point mutants made to interrogate nucleotide base constraints at each position of the heptaloop sequence found that any base (N) is tolerable at the third and fourth positions, but specific identities at the third position result in improved basal activity (C>A/U>G). The fifth and sixth positions accept both purine bases (A, G) and retain activity similar to that of the L2b8 parent; however, an adenine base (A) in the fifth position results in improved basal activity. Point mutants verified the requirement of a guanine base (G) in the seventh position.

For the triloop sequence, point mutants indicate that any combination of bases in the first and seventh positions resulting in canonical Watson-Crick base pairing (AU, GC; order independent) results in improved basal activity. The bases in the second and sixth positions must result in a Watson-Crick GU wobble pair, where this requirement is sensitive to identity and order (i.e., standard Watson-Crick pairing abolishes improved activity). The point mutants indicate that the third position is able to accept any nucleotide base (N), whereas a guanine base (G) is required at the fourth and fifth positions to retain improved activity relative to the L2b8 parent device.

FIG. 14. Representative cleavage assays for measuring cleavage rate constants (k) for ribozyme-based devices and controls. A representative assay is shown for each device in the absence and presence (0 and 5 mM, respectively) of theophylline: L2b1 (A), L2b5 (B), L2b8 (C), L2b8-a1 (D), L2b8-a14 (E), L2b8-a1-t41 (F), and sTRSV ribozyme (G). Bands for both the cleaved products (5′C, 3′C) and the full-length uncleaved substrate (UC) are shown for L2b1. For subsequent devices, the shorter 5′C product is omitted from the inset image for clarity. Methods used to prepare full-length, uncleaved RNA transcripts and conditions of the cleavage assays are detailed in the Materials and Methods section of the main text. Briefly, RNA was heated to 95° C. for 5 min and cooled to 37° C. in a secondary structure refolding buffer (100 mM NaCl, 50 mM Tris-HCl (pH 7.5)). A zero time point aliquot was removed prior to initiating the reaction with addition of MgCl₂ to a final concentration of 500 μM. Reactions were quenched at the indicated time points. At least seven time points were taken in each cleavage assay to capture the cleavage dynamics of RNA devices exhibiting different cleavage kinetics. A black vertical bar was used to denote time points run on different gels. Phosphorimaging analysis of relative levels of the UC, 5′C, and 3′C bands was used to determine the fraction cleaved at each time point (F₁). The fraction cleaved at the beginning (F₀) and end of reaction (F_{∞}) varied between assays, but all assays were well-fit to the single exponential equation (R²>0.95):

Fₜ=F₀+(F_{∞}−F₀)×(1−e^{-kt})

The black and red fit lines represent assays performed at 0 and 5 mM theophylline, respectively. The cleavage rate constant value (k) was determined for each assay. The reported k for each device and theophylline assay condition is the mean and standard deviation of at least three independent experiments (H).

FIG. 15. Increased input ligand concentration results in higher device ON states. Increasing ligand concentration to 40 mM theophylline (theo) increases device activation ratio (AR) compared to 5 mM theophylline. Gene-regulatory activities are reported as the geometric mean of the GFP fluorescence of the indicated sample normalized to that of a positive control (sTRSV Contl, a noncleaving sTRSV ribozyme with a scrambled core) that is grown under identical ligand conditions and is set to 100%. Reported values are the mean and standard deviation of at least three independent experiments. The activation ratio (AR), determined as the ratio of gene expression levels in the presence and absence of theophylline (theo), is indicated for each device.

FIG. 16. Quantification of nonspecific ligand effect on fluorescence intensity and cell viability. (A) Unnormalized data for GFP fluorescence, mCherry fluorescence, and cell viability for the positive control construct (sTRSV Contl, a noncleaving sTRSV ribozyme with a scrambled core) in all ligand conditions tested. Fluorescence values are reported as the geometric mean of the GFP or mCherry fluorescence of sTRSV Contl. Cell viability is reported as the percentage of cells included in the DAPI-(−) gate. Mean and standard deviation of three independent experiments are indicated. The ratio of fluorescence levels or cell viability percentages in the presence and absence of theophylline (theo) or tetracycline (tc) is indicated for each ligand condition. Variability in absolute GFP or mCherry signal among replicates for an individual ligand condition is due to differences in instrument calibration between experiments. Values for each replicate pair in the absence and presence of ligand were obtained in the same experiment with identical instrument calibration. (B) Scatter plots for sTRSV Contl under each ligand condition from a single representative experiment.

FIG. 17. Component swapping demonstrates modularity of the optimized actuator components. Replacement of the loop I sequence with the a1 or a14 sequence decreases basal activity in all devices tested, including theophylline (theo)-responsive ON (A, B) and OFF (C) switches and a tetracycline-responsive ON switch (D). Switching activity is maintained in most devices. Notably, replacement of the loop I sequence in the sTRSV ribozyme (E) significantly increases basal activity. Gene-regulatory activities are reported as the geometric mean of the GFP fluorescence of the indicated sample normalized to that of a positive control (sTRSV Contl, a noncleaving sTRSV ribozyme with a scrambled core) that is grown under identical ligand conditions and is set to 100%. Reported values are the mean and standard deviation of at least three independent experiments. The activation ratio (AR), determined as the ratio of gene expression levels in the presence and absence of theophylline (theo) (A, B, C) or of tetracycline (D), is indicated for each device.

FIG. 18. The activities of the optimized actuator components are maintained in the context of the newly selected transmitter components. For devices isolated from the tN11 sort (L2b8-t11 and L2b8-t47), replacement of the actuator loop I sequence with the a1 sequence results in lower basal activity. For L2b8-a1-t41, which was isolated from the a1-tN11 sort, replacement of the a1 sequence with the loop I sequence of L2b8 results in higher basal activity. Gene-regulatory activities are reported as the geometric mean of the GFP fluorescence of the indicated sample normalized to that of a positive control (sTRSV Contl, a noncleaving sTRSV ribozyme with a scrambled core) that is grown under identical ligand conditions and is set to 100%. Reported values are the mean and standard deviation of at least three independent experiments. The activation ratio (AR), determined as the ratio of gene expression levels in the presence and absence of theophylline (theo), is indicated for each device.

FIG. 19. Secondary structure analysis indicates similarities between engineered RNA devices and natural hammerhead ribozymes (HHRzs). The HHRz invariant nucleotide bases are boxed in black, and the cleavage site is denoted by an arrow in all structures. In RNA device structures (A, B, D), nucleotide bases of the transmitter and aptamer domains are boxed in grey and olive green respectively. Using the common numbering system for HHRz with apical loops (Hertel et al., “Numbering system for the hammerhead,” Nucleic Acids Res. (1992) 20: 3252), nucleotide bases highlighted in cyan (L1.1 U) and red (L1.6 U) are those conforming to the common HHRz loop I motif, UNₘYN, where m is typically 3 or 4 bases. Common loop II motif nucleotide bases, (RNA), are highlighted in yellow (L2.1 G) and orange (L2.1 A), where n is usually 2 or 4 bases (Dufour et al., “Structure-function analysis of the ribozymes of chrysanthemum chlorotic mottle viroid: a loop-loop interaction motif conserved in most natural hammerheads,” Nucleic Acids Res. (2009) 37: 368-381). R and Y denote purine and pyrimidine bases respectively. (A) The HHRz core of the L2b8 parent device forms a predicted GU base pair at L2.2-L2.3 due to transmitter-sensor integration into loop II when analyzed by RNAstructure folding software (Mathews et al., “Incorporating chemical modification constraints into a dynamic programming algorithm for prediction of RNA secondary structure,” Proc. Nat'l Acad. Sci. USA (2004) 101:7287-7292). HHRz loop sequence similarities exist between a device containing the variant heptaloop consensus sequence selected in this study (B) and the newt HHRz (C). In loop I, both HHRz structures have AU in the first two positions 5′ from the catalytic core (boxed in green) and RG in last two positions (boxed in brown). In loop II, both structures have a G nucleotide not constrained in a Watson-Crick base pair (circled in cyan). Loop sequence similarities also exist between a device containing the variant triloop consensus sequence (D) and the CarSVRNA(−) HHRz (E). Both loop I sequences in these HHRzs contain a triloop with a GU base pair closing the loop (circled in purple) and accommodate GG in the first two positions 5′ from the catalytic core (boxed in green). In loop II, both HHRz structures have L2.1 G and L2.4 A nucleotides not constrained in Watson-Crick base pairs, available for loop I-loop II interaction, circled in cyan (Dufour et al., supra). Secondary structures rendered using VARNA software (Darty et al., “VARNA: Interactive drawing and editing of the RNA secondary structure,” Bioinformatics (2009) 25: 1974-1975).

FIG. 20. Plasmid map of pCS1748. RNA devices are inserted in the 3′ UTR of yEGFP3.

FIG. 21. Library design for sN10 sensor, aN7 actuator, and tN11 and a1-tN11 transmitter libraries. Predicted secondary structures of the active ribozyme conformation with unbound theophylline aptamer (left) and the inactive ribozyme conformation with bound theophylline aptamer (right) are shown. The sequence for the L2b8 parent device is shown. Nucleotides that comprise the sensor, actuator, and transmitter components are colored blue, black, and yellow, respectively. Theophylline is represented by the red polygon. Filled circles within each component indicate nucleotides randomized to generate respective device libraries. Secondary structures were predicted by RNAstructure folding software (Mathews et al., supra) and rendered using VARNA software (Darty et al, supra).

FIG. 22. Comparison of efficiency between single-color and two-color FACS-based strategy. (A) Three fractions of cells that exhibit low activity were collected (left panel). Only fraction A maintained a low level of GFP expression after the fractions were regrown (right panel). (B) Cells that exhibit GFP fluorescence levels below a cell population containing a positive GFP control construct lacking an Rnt1p hairpin module were collected (left panel). The collected cells maintained a low level of GFP expression when regrown (right panel).

### DETAILED DESCRIPTION

Methods of evaluating a gene-regulatory element, including libraries of known or candidate gene-regulatory elements, are provided. Aspects of the methods include cytometrically analyzing a cell comprising a gene-regulatory element construct, e.g., a plasmid, having an activity reporter comprising a first signal reporter domain which produces a first signal operatively coupled to a gene-regulatory element of interest; and a noise reporter comprising a second signal reporter which produces a second signal that is distinguishable from the first signal to obtain the first and second signals. The first signal is then normalized with the second signal to obtain a normalized activity signal, which normalized activity signal is then employed to evaluate the gene-regulatory element. Also provided are reagents, systems and kits that find use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Unless defined otherwise, all technical and scientific terms used herein have the same meaning as commonly understood by one of ordinary skill in the art to which this invention belongs.

Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present invention and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing embodiments of the invention, aspects of embodiments of the methods will be described first in greater detail. Next, embodiments of devices and systems that may be used in practicing methods of the inventions, as well as kits of reagents, are reviewed.

### Methods

As summarized above, aspects of the invention include methods of evaluating a gene-regulatory element. The term “evaluating” means assessing one or more parameters or characteristics of a gene-regulatory element, and refers to any form of measurement of such a parameter or characteristic. Accordingly, evaluating as used herein includes determining whether a gene-regulatory element of interest has a given property. The terms “determining,” “measuring,” and “assessing,” and “assaying” are used interchangeably and include both quantitative and qualitative determinations. Assessing may be relative or absolute. “Assessing the presence of includes determining the amount of something present, as well as determining whether it is present or absent. As such, aspects of the invention include methods of determining one or more qualities of a gene-regulatory element. A variety of different parameters may be evaluated, where examples of such are described in greater detail below.

As summarized above, aspects of the invention include methods of evaluating gene-regulatory elements. Gene-regulatory elements that may be evaluated in methods, e.g., as described herein, are regulatory elements that are composed of one or more components, at least one of which is a nucleic acid component. Gene-regulatory elements of interest may vary, and may encode more basic functions, such as sensing, information transmission, and actuation. Examples of gene-regulatory elements of interest include, but are not limited to: RNA control devices and components thereof, e.g., actuators (such as ribozymes), transmitters, sensors, etc.; promoters; RNase substrates, and the like. Examples of embodiments of gene-regulatory elements of interest are further described in greater detail below. In some instances, the gene-regulatory elements are ligand-responsive gene-regulatory elements, such that their activity is modulated (e.g., they are active or inactive) bythe presence of a specific ligand, such as a small molecule (e.g., theophylline, tetracylcine, etc.), a protein (e.g., signaling proteins, such as β-catenin; transcription factors, such as NF-kB; an enzymes, such as an RNA polymerase or a DNase); Peptide, lipid, metal ion, etc. As used herein, the phrase gene-regulatory element refers to both elements that exhibit gene-regulatory activity, as well as putative or candidate gene-regulatory elements, i.e., elements that are at least suspected of exhibiting gene-regulatory activity, such as gene-regulatory elements that may be found in a cellular library of candidate gene regulatory elements. As such, the phrase gene-regulatory elements includes elements that have not yet been demonstrated to possess gene-regulatory activity, but are suspected of possibly possessing such activity and therefore suitable for screening for gene-regulatory activity.

In evaluating a gene-regulatory element according to methods described herein, a cell comprising a gene-regulatory element construct is cytometrically analyzed. The gene-regulatory construct that is present in the cell is a nucleic acid construct that includes both an activity reporter and a noise reporter. Both the activity reporter and noise reporter are functional modules, i.e., expression cassettes. The term “expression cassette” as used herein refers to a nucleic acid region or domain that includes at least a promoter and a coding sequence, where the disparate components are operatively coupled or linked to each other such that, under appropriate conditions (e.g., intracellular conditions in the presence of any expression required factors, amino acids and enzymes, etc.), the coding sequence may be expressed to produce a product, e.g., a protein. A given expression cassette may include additional elements, e.g., termination sequences, etc., as desired.

The activity reporter is an expression cassette that provides a signal which corresponds to the activity of a gene-regulatory element of interest, at least a component of which is part of the activity reporter. By corresponds is meant that the observed signal is proportional to the expression level of a signal reporter coding sequence of the reporter. The activity reporter includes at least a first signal reporter operatively coupled to a gene-regulatory element of interest. Depending on the particular gene-regulatory element of interest, the activity reporter may further include additional components, e.g., a promoter, a termination sequence, etc. (e.g., as described in greater detail below).

The first signal reporter is a coding sequence for a member of a first signal producing system. A given signal producing system as described herein may include a single component, e.g., a fluorescent protein, or two or more components, e.g., an enzyme and a substrate that is converted by the enzyme to a detectable product. Accordingly, the first signal producing system may vary, where examples of signal producing systems include, but are not limited to: fluorescent proteins, enzymes that convert a substrate to a detectable product, etc.

A variety of coding sequences for fluorescent proteins may be employed. As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak in the emission spectrum at 510 nm or about 510 nm. A variety of FPs that emit at various wavelengths are known in the art. FPs of interest include, but are not limited to, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein. As used herein, Aequorea GFP refers to GFPs from the genus Aequorea and to mutants or variants thereof. Such variants and GFPs from other species, such as Anthozoa reef coral, Anemonia sea anemone, Renilla sea pansy, Galaxea coral, Acropora brown coral, Trachyphyllia and Pectimidae stony coral and other species are well known and are available and known to those of skill in the art. Exemplary GFP variants include, but are not limited to BFP, CFP, YFP and OFP. Examples of florescent proteins and their variants include GFP proteins, such as Emerald (Invitrogen, Carlsbad, Calif.), EGFP (Clontech, Palo Alto, Calif.), Azami-Green (MBL International, Woburn, Mass.), Kaede (MBL International, Woburn, Mass.), ZsGreen1 (Clontech, Palo Alto, Calif.) and CopGFP (Evrogen/Axxora, LLC, San Diego, Calif.); CFP proteins, such as Cerulean (Rizzo, Nat. Biotechnol. 22(4):445-9 (2004)), mCFP (Wang et al., PNAS USA. 101(48):16745-9 (2004)), AmCyanl (Clontech, Palo Alto, Calif.), MiCy (MBL International, Woburn, Mass.), and CyPet (Nguyen and Daugherty, Nat. Biotechnol. 23(3):355-60 (2005)); BFP proteins such as EBFP (Clontech, Palo Alto, Calif.); YFP proteins such as EYFP (Clontech, Palo Alto, Calif.), YPet (Nguyen and Daugherty, Nat. Biotechnol. 23(3):355-60 (2005)), Venus (Nagai et al., Nat. Biotechnol. 20(1):87-90 (2002)), ZsYellow (Clontech, Palo Alto, Calif.), and mCitrine (Wang et al., PNAS USA. 101(48):16745-9 (2004)); OFP proteins such as cOFP (Strategene, La Jolla, Calif.), mKO (MBL International, Woburn, Mass.), and mOrange; and others (Shaner N C, Steinbach P A, and Tsien R Y., Nat. Methods. 2(12):905-9 (2005)). As used herein, red fluorescent protein, or RFP, refers to the Discosoma RFP (DsRed) that has been isolated from the corallimorph Discosoma (Matz et al., Nature Biotechnology 17: 969-973 (1999)), and red or far-red fluorescent proteins from any other species, such as Heteractis reef coral and Actinia or Entacmaea sea anemone, as well as variants thereof RFPs include, for example, Discosoma variants, such as monomeric red fluorescent protein 1 (mRFP1), mCherry, tdTomato, mStrawberry, mTangerine (Wang et al., PNAS USA. 101(48):16745-9 (2004)), DsRed2 (Clontech, Palo Alto, Calif.), and DsRed-T1 (Bevis and Glick, Nat. Biotechnol., 20: 83-87 (2002)), Anthomedusa J-Red (Evrogen) and Anemonia AsRed2 (Clontech, Palo Alto, Calif.). Far-red fluorescent proteins include, for example, Actinia AQ143 (Shkrob et al., Biochem J. 392(Pt 3):649-54 (2005)), Entacmaea eqFP611 (Wiedenmann et al. Proc Natl Acad Sci USA. 99(18):11646-51 (2002)), Discosoma variants such as mPlum and mRasberry (Wang et al., PNAS USA. 101(48):16745-9 (2004)), and Heteractis HcRed1 and t-HcRed (Clontech, Palo Alto, Calif.).

As mentioned above, also of interest as reporters are coding sequences for enzymes that convert a substrate to a detectable, e.g., fluorescent product. Examples of such enzymes include, but are not limited to: luciferases, e.g., firefly luciferase (de Wet et al. (1987) Mol. Cell. Biol. 7: 725-737), Renilla luciferase from Renilla renformis (Lorenz et al. (1991) PNAS USA 88: 4438-4442), click beetle luciferase (CBG99; Wood et al. (1989) Science 244(4905): 700-2); β-galactosidase (LacZ); β-glucuronidase (gusA); xanthineguanine phosphoribosyltransferase (XGPRT), etc.

In addition to the activity reporter, the gene-regulatory construct also includes a noise reporter. As with the activity reporter, the noise reporter is an expression cassette that includes a promoter operatively coupled to a coding sequence, where the coding sequence of the noise reporter is a second signal reporter that encodes a product which is a member of a second signal producing system, where the second signal producing system produces a detectable signal that is distinguishable from the detectable signal produced by the first signal producing system. By distinguishable is meant that the signals produced by the first and second signal producing systems may be recognized as distinct from each other, such that they can be known as different signals. As such, the signals are not identical or the same. Examples of distinguishable signals are different fluorescent emission maxima. As with the first signal producing system, the second signal producing system may vary, where examples of suitable second signal producing systems include, but are not limited to: fluorescent proteins, enzymes that convert a substrate to a detectable product, etc. In certain embodiments, the second signal producing system is the same type of signal producing system as the first signal producing system. In certain embodiments, the first and second signal producing systems are first and second fluorescent proteins and therefore the first and second signal reporters of the activity and noise reporters are coding sequences for different first and second fluorescent proteins.

In those embodiments where the first and second signal reporters encode first and second fluorescent proteins, the first and second fluorescent proteins will have emission maxima that are sufficiently different to provide for adequate differentiation of the respective signals. As such, the difference between emission maxima may, in some instances, be 25 nm or greater, such as 50 nm or greater, including 75 nm or greater. Any suitable pair of fluorescent proteins may be employed, where the fluorescent proteins may or may not be excitable at the same wavelength. Suitable pairs of interest include, but are not limited to: green fluorescent proteins with red fluorescent proteins, cyan fluorescent proteins with yellow fluorescent proteins, etc.

With respect to the activity and noise reporters, these two functional modules may have the same or different promoters. A variety of different types of promoters may be employed, where promoters of interest include both constitutive and inducible promoters. Promoters for a given screen will be selected based, at least in part, on the host cell in which the gene-regulatory construct is to be evaluated. For example, where the host cell is a yeast cell, yeast operative promoters will be employed. Examples of yeast operative promoters of interest include, but are not limited to: TDH3, TEF1, PGK1, CYC1, GAL10, ATF2, TIR1, ARH1 and ADE2, and the hybrid promoter GAL10-CYC1.

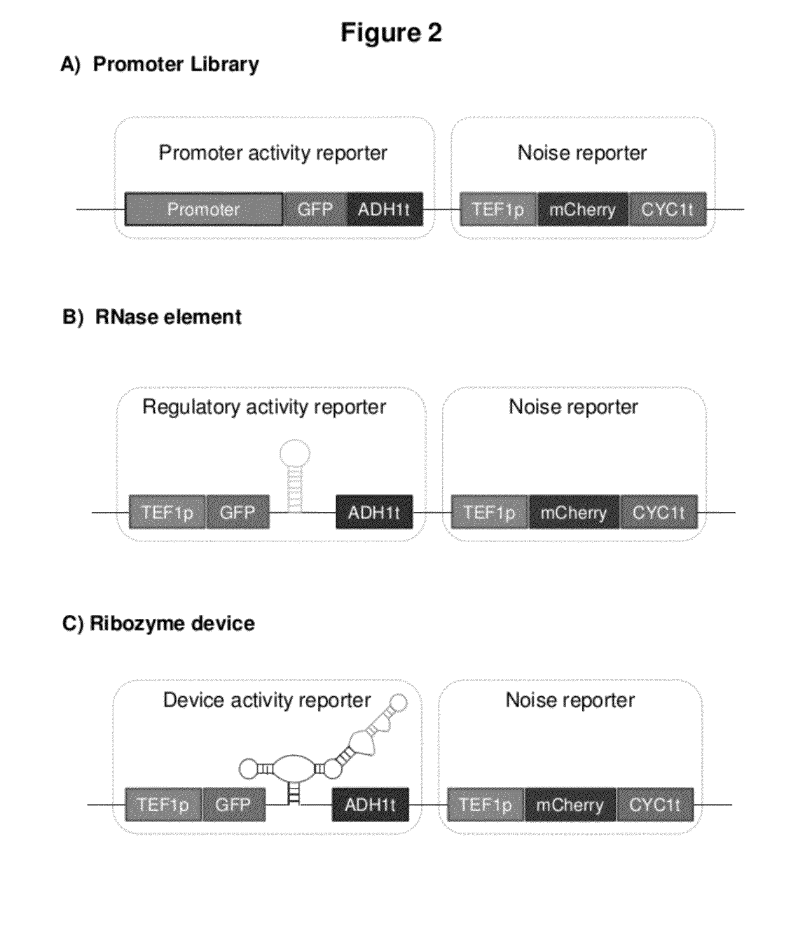

As mentioned above, the gene-regulatory element that is evaluated using methods of the invention may vary. FIG. 2 illustrates examples of gene-regulatory element constructs suitable for evaluating various types of gene-regulatory elements. FIG. 2A illustrates a gene regulatory element suitable for evaluating a promoter. In FIG. 2A, the gene-regulatory element construct includes an activity promoter labeled Promoter Activity Reporter, which includes a promoter of interest to be evaluated operably linked to a coding sequence for GFP (which is the first signal reporter) and a termination sequence, i.e., ADH1t. The noise reporter of the construction shown in FIG. 2A includes the constitutive yeast promoter TEF1p operatively coupled to a coding sequence for mCherry (which is the second signal reporter) and a second termination sequence, CYC1t. While the termination sequences are different in the activity and noise reporters, they can, in some instances, be the same. The promoter element to be assayed may vary widely, where examples of suitable promoters include, but are not limited to, the following specific promoters and mutants thereof. The promoters may be constitutive or inducible. Constitutive promoters include, but are not limited to, immediate early cytomegalovirus (CMV) promoter, herpes simplex virus 1 (HSV1) immediate early promoter, SV40 promoter, lysozyme promoter, early and late CMV promoters, early and late HSV promoters, beta-actin promoter, tubulin promoter, Rous-Sarcoma virus (RSV) promoter, and heat-shock protein (HSP) promoter. Inducible promoters include tissue-specific promoters, developmentally-regulated promoters and chemically inducible promoters. Examples of tissue-specific promoters include the glucose-6-phosphatase (G6P) promoter, vitellogenin promoter, ovalbumin promoter, ovomucoid promoter, conalbumin promoter, ovotransferrin promoter, prolactin promoter, kidney uromodulin promoter, and placental lactogen promoter. Examples of developmentally-regulated promoters include the homeobox promoters and several hormone induced promoters. Examples of chemically-inducible promoters include reproductive hormone induced promoters and antibiotic-inducible promoters such as the tetracycline-inducible promoter and the zinc-inducible metallothionine promoter. Such constructs can readily be employed to evaluate a library of promoter variants. Promoter libraries can be generated through PCR mutagenesis based on an endogenous promoter, such as described above. The regulatory activities of the promoter libraries are measured by the first fluorescence reporter (GFP) and normalized by the second fluorescence reporter (mCherry), e.g., as described greater detail below. The sorting gate can be set based on other control elements (i.e., RNA devices or components) that have been previously characterized (e.g., by a control 2-color construct identical to the gene-regulatory element construct of interest but for the presence of a gen-regulatory element of known characteristics) to isolate promoter variants that exhibit the desired activities.

Another gene-regulatory element construct of interest is one configured to evaluate an RNase control element, e.g., as illustrated in FIG. 2B. In FIG. 2B, the activity and noise reporters of the construct include the same promoter, TEF1p, operatively coupled to different fluorescent protein coding sequences and termination sequences, which are the same as those described above in connection with the construct shown in FIG. 2A. The activity reporter further includes a RNase hairpin loop structure, which serves as a substrate for an RNase. Such constructs may be employed to evaluate libraries of different hairpin loop structures and/or RNases for their suitability as gene-regulatory elements.

Another gene-regulatory element construct of interest is one configured to evaluate a ribozyme device, e.g., as illustrated in FIG. 2C. In FIG. 2C, the activity and noise reporters of the construct include the same promoter, TEF1p, operatively coupled to different fluorescent protein coding sequences and termination sequences, which are the same as those described above in connection with the construct shown in FIG. 2A. The activity reporter further includes a ribozyme-based regulatory element. The ribozyme-based regulatory element may be a member of a modular RNA control device that includes an actuator (e.g., hammerhead ribozyme), sensor and transmitter components, e.g., as described in Win & Smolke, Proc. Nat'l Acad. Sci. USA (2007) 104: 14283-14288 and Win & Smolke, Science (2008) 322:456-460. Such constructs may be employed to evaluate libraries of different RNA control devices and components thereof, e.g., actuators, sensors, transmitters, etc.

In some instances, a gene-regulatory system may be ligand responsive, e.g., as described above. Accordingly, in some embodiments the methods are methods of screening a ligand, or library of ligands, for activity in a gene-regulatory system. In such instances, the gene-regulatory construct may include one component of the ligand responsive gene-regulatory system, and cell harboring the construct contacted with the ligand in order to evaluate the ligand component of the gene-regulatory system. A library of candidate ligands may be evaluated in such a manner. The candidate ligand may be chosen from a variety of different molecules. A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As desired, the gene-regulatory element construct including both the activity and noise reporters (such as described above) may be chromosomally integrated or episomally maintained in the cell. When chromosomally integrated, the construct is stably part of a chromosome of a host cell. When episomally maintained, the construct is present on a vector, e.g., a plasmid, an artificial chromosome, e.g. BAC or YAC, that is not part of a host cell's chromosome. While the length of the gene-regulatory construct may vary, in some instances the length ranges from 1 kb to 50 kb, such as 4 kb to 25 kb, including 4 kb to 10 kb.

In evaluating gene-regulatory elements as described herein, the gene-regulatory element, e.g., as described above, is provided in a host cell. The host cell that includes the gene-regulatory element may vary greatly. Host cells may be single cells, cell lines or components of a multi-cellular organism. In some instances, the cell is a eukaryotic cell. Some examples of specific cell types of interest include, but are not limited to: bacteria, yeast (e.g., S. cerevisiae, S. pombe, P. pastoris, K. lactis, H. polymorpha); fungal, plant and animal cells. Host cells of interest include animal cells, where specific types of animal cells include, but are not limited to: insect, worm or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

The host cells may be prepared using any convenient protocol, where the protocol may vary depending on nature of the host cell, etc. Where desired, vectors, such as viral vectors, may be employed to engineer the cell to contain the gene-regulatory element construct, as desired (see e.g., retroviral and adenoviral vectors, such as provided commercially by Clontech Laboratories, Mountain View, Calif.). Depending on the nature of the host cell and/or gene-regulatory element construct, protocols of interest may include electroporation, particle gun technology, calcium phosphate precipitation, transfection agent mediated introduction, direct microinjection, viral infection and the like. The choice of method is generally dependent on the type of cell being transformed and the circumstances under which the transformation is taking place. A general discussion of these methods can be found in Ausubel, et al, Short Protocols in Molecular Biology, 3rd ed., Wiley & Sons, 1995. After the vector nucleic acids comprising the gene-regulatory element construct of interest have been introduced into a cell, the cell may be incubated, e.g., at 37° C., sometimes under selection, for a period of about 1-24 hours.

As mentioned above, in some instances the gene-regulatory element is a ligand-responsive gene-regulatory element. In such instances, the cell comprising the gene-regulatory construct will be manipulated in some manner to provide the requisite ligand. For example, where the ligand is a small molecule, e.g., theophylline, the cell may be contact with a quantity of the small molecule. Alternatively, where the ligand is encoded by a genetic element in the cell, e.g., a coding sequence for an enzyme, the cell may be contacted with an inducer of expression of the genetic element. Any convenient protocol for providing the ligand in the cell may be employed.

Following production of the host cell and provision of any requisite ligand, e.g., as described above, the host cell is then cytometrically analyzed to obtain signals from the first and second reporters. Flow cytometry is a well-known methodology using multi-parameter data for identifying and distinguishing between different particle types (i.e., particles that vary from one another terms of label (wavelength, intensity), size, etc., in a fluid medium. In flow cytometrically analyzing the cells prepared as described above, a liquid medium comprising the cells is first introduced into the flow path of the flow cytometer. When in the flow path, the cells in the sample are passed substantially one at a time through one or more sensing regions, where each of the cells is exposed separately individually to a source of light at a single wavelength (or in some instances two or more distinct sources of light having different wavelengths) and the resultant signals, e.g., first and second signals, are separately recorded for each cell.

More specifically, in a flow cytometer, the cells are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 535 nm, 635 nm, and the like.

In series with a sensing region, detectors, e.g., light collectors, such as photomultiplier tubes (or “PMT”), image collectors (e.g., in the form of charge-coupled devices (CODs)), etc., are used to record light that passes through each cell (generally referred to as forward light scatter), light that is reflected orthogonal to the direction of the flow of the cells through the sensing region (generally referred to as orthogonal or side light scatter) and fluorescent light emitted from the cells, if it is labeled with fluorescent marker(s), as the cells passes through the sensing region and is illuminated by the energy source. Each type of data that is obtained, e.g., forward light scatter (or FSC), orthogonal light scatter (SSC), and fluorescence emissions (FL1, FL2, etc.) and image, etc., comprise a separate parameter for each cell (or each “event”).

Flow cytometers further include data acquisition, analysis and recording means, such as a computer, wherein multiple data channels record data from each detector for the data emitted by each cell as it passes through the sensing region. The purpose of the analysis system is to classify and count cells wherein each cell presents itself as a set of digitized parameter values.

The flow cytometer may be set to trigger on a selected parameter in order to distinguish the cells of interest from background and noise. “Trigger” refers to a preset threshold for detection of a parameter. It is typically used as a means for detecting passage of cells through the laser beam. Detection of an event which exceeds the threshold for the selected parameter triggers acquisition of light scatter and fluorescence data for the cell. Data is not acquired for cells or other components in the medium being assayed which cause a response below the threshold. The trigger parameter may be the detection of forward scattered light caused by passage of a cell through the light beam. The flow cytometer then detects and collects the light scatter and fluorescence data for a cell.

It will be understood by those of skill in the art that the stated expression levels reflect detectable amounts of the marker protein. A cell that is negative for staining (the level of marker specific reagent is not detectably different from a negative control) may still express minor amounts of the marker. And while it is commonplace in the art to refer to cells as “positive” or “negative” for a particular marker, actual expression levels are quantitative traits. The number of molecules on the cell surface can vary by several logs, yet still be characterized as “positive”.

Although the absolute level of staining may differ with a particular fluorochrome and reagent preparation, the data can be normalized to a control. Normalization refers to the comparison of one or more datasets to a common variable in order to account for and negate the variability between the datasets, thus allowing underlying characteristics of the datasets to be compared. In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control may utilize a substrate having a defined density of marker on its surface, for example a fabricated bead or cell line, which provides the positive control for intensity.

An aspect of the methods is that they include a step of normalizing the cytometrically obtained first signal with the cytometrically obtained second signal to obtain a normalized activity signal. As such, for each analyzed cell, the first signal detected by the flow cytometer is normalized to a the second signal obtained by the flow cytometer in order to obtain a true activity signal for the gene-regulatory element that is substantially free of a noise component. Any convenient protocol and algorithm may be employed for normalizing the first signal with the second signal, where suitable algorithms include, but are not limited to compensation and gating protocols, e.g., as employed in the FlowJo software, and the like.

For example, the cells may be analyzed by one or more “gating” steps based on the data collected for the entire population (or desired portion thereof) being analyzed. To select an appropriate gate, the data is plotted so as to obtain the best separation of subpopulations possible. For example, in a first gating step, gates may be defined by forward light scatter (FSC) vs. side (i.e., orthogonal) light scatter (SSC) on a two dimensional dot plot. Alternatively or in addition, a gating step may be performed with two color analysis. As one example, appropriate gates may be set based on any RNA element previously characterized for its regulatory activity. The flow cytometer operator then selects the desired subpopulation of cells (i.e., those cells within the gate) and excludes cells which are not within the gate. Where desired, the operator may select the gate by drawing a line around the desired subpopulation using a cursor on a computer screen. Only those cells within the gate are then further analyzed by plotting the other parameters for these cells, such as fluorescence. By using normalized activity signals as described herein, high resolution of different cell populations with minimal, if any overlap, is obtained, providing for highly quantitative evaluation of a given gene-regulatory element.

Flow cytometry, or FACS, can also be used to separate or “sort” individual cells or cell populations based on the intensity of marker expression or binding to a specific reagent, as well as other parameters such as cell size and light scatter, to create enriched populations of cells, e.g. cells comprising a gene-regulatory construct of interest, e.g. to create enriched device libraries. The enriched populations will be 75% or more, 80% or more, 85% or more, 90% or more of the desired cells, in some instances, 95% or more of the desired cells, e.g. 98%, 99% or 100% of the desired cells. In other words, the composition will be a substantially pure composition of cells of interest. Cells of interest may be sorted into a single vessel for propagation and/or analysis. Alternatively, cells may be sorted into multiple vessels, e.g. one cell per vessel, e.g. into wells of a tissue culture plate, for propagation and/or analysis, e.g. a clonal populations. The sorted cells may be used immediately, or may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The sorted cells may be cultured in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive, e.g. molecules capable of promoting survival, growth and/or differentiation of cells, either in culture or in the intact tissue, e.g. through specific effects on a transmembrane receptor.

Any flow cytometer that is capable of obtaining both the morphometric and biomarker/non-specific cell data, e.g., as described above, from the same aliquot of a liquid sample may be employed. Of interest are those flow cytometer systems described in U.S. Pat. Nos. 6,211,955, 6,249,341, 6,256,096, 6,473,176, 6,507,391, 6,532,061, 6,563,583, 6,580,504, 6,583,865, 6,608,680, 6,608,682, 6,618,140, 6,671,044, 6,707,551, 6,763,149, 6,778,263, 6,875,973, 6,906,792, 6,934,408, 6,947,128, 6,947,136, 6,975,400, 7,006,710, 7,009,651, 7,057,732, 7,079,708, 7,087,877, 7,190,832, 7,221,457, 7,286,719, 7,315,357, 7,450,229, 7,522,758, 7,567,695, 7,610,942, 7,634,125, 7,634,126, 7,719,598; the disclosures of which are herein incorporated by reference.

Flow cytometric analysis of the cells, as described above, yields qualitative and quantitative information about the activity of the gene-regulatory element of interest in the cells. A variety of different gene-regulatory element parameters may be evaluated using methods described herein. Gene-regulatory element parameters that may be evaluated include, but are not limited to: basal activity, activation ratio, dynamic range, EC₅₀, ligand saturated activity, and the like. The basal activity is a measure of regulatory stringency, which is set by the gene expression level in the absence of the input ligand. Activation ratio is a measure of input responsiveness, which is set by the ratio of expression level in the presence to that in the absence of input ligand.

Aspects of the invention include the high-throughput evaluation of two or more distinct gene-regulatory elements, such as 2 or more, 5 or more, 10 or more, 25 or more, including larger libraries of gene-regulatory elements, e.g., libraries of 50 or more, 100 or more, 10³ or more, 10⁴ or more, 10⁵ or more, 10⁶ or more etc. For example, in some instances a library of putative or candidate gene-regulatory elements, e.g., RNA control device elements (actuators, sensors, transmitters or ligands), RNase elements, promoters, etc., may be transfected into a host cell, e.g., a yeast strain, and the resultant cellular library (following any desired step of ligand provision) may then be flow cytometrically analyzed, e.g., as described above. In such screens the gene-regulatory constructs of the library members may harbor the same or different component of the gene-regulatory system of interest. For example, where the gene-regulatory element of interest is an actuator, the library members may differ from each other in terms of the gene-regulatory element on the construct, such that the cellular library that is cytometrically analyzed will include a cell having a construct that differs from the construct of another cell by including a different gene-regulatory element in the activity reporter. In other embodiments, the gene-regulatory constructs may be identical in the library members, and the library members will differ from each other in terms of another component of the system, e.g., the transmitter, ligand, etc. Such libraries will be employed in screens of library system components such as transmitter libraries, ligand libraries, etc.

Libraries can be generated through a number of different means, including DNA synthesis of randomized libraries and PCR mutagenesis. In some instances, libraries of RNA components (control elements) and RNA devices (sensing-actuation elements), which are composed of several components, are evaluated. When generating RNA device libraries, one may randomize specific components within the device, such as an actuator component or sensor component.

### Devices and Systems

Aspects of the invention further include devices and systems for use in practicing the methods, e.g., as described herein. Systems of interest include a flow cytometer configured to assay a liquid sample for both a first and second fluorescent signal and then normalize the first signal with the second signal to obtain an normalized activity signal, e.g., as described above. Flow cytometers of interest include, but are not limited, those devices described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of which are herein incorporated by reference.

In some instances, the flow cytometer includes: a flow channel; at least a first light source configured to direct light to an assay region of the flow channel (where in some instances the cytometer includes two or more light sources); a first detector configured to receive light of a first wavelength from the assay region of the flow channel; and a second detector configured to receive light of a second wavelength from the assay region of the flow channel. Such a cytometer would have at least two detection channels. In some instances, the device may include more than two detection channels, e.g., 3 or more, 4 or more, 5 or more, 10 or more, etc.

Aspects of the invention further include a signal processing module configured to receive the first and second signals and normalize the first and second signals to provide the normalized activity signal. The signal processing module may further be configured to provide an evaluation of a gene-regulatory element based on the obtained normalized activity signal. The signal processing module may be integrated into the cytometer as a single device, or distributed from the cytometer where the signal processing module and cytometer are in communication with each other, e.g., via a wired or wireless communication protocol.

Accordingly, aspects of the invention further include systems, e.g., computer-based systems, which are configured to evaluate a gene-regulatory element, e.g., as described above. A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Embodiments of the subject systems include the following components: (a) a communications module for facilitating information transfer between the system and one or more users, e.g., via a user computer, as described below; and (b) a processing module for performing one or more tasks involved in the quantitative analysis methods of the invention. In certain embodiments, a computer program product is described comprising a computer usable medium having control logic (computer software program, including program code) stored therein. The control logic, when executed by the processor of the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In some instances, the systems may further include a cellular sample, where the cellular sample is prepared as described above, e.g., by providing cells containing a gene-regulatory construct (and optionally a ligand to which the gene-regulatory element of the construct is responsive).

### Utility

The subject methods and systems find use in a variety of different applications where evaluation of a gene-regulatory element is desired. Such applications include the engineering of biological systems to control the ability of such systems to process information within cellular networks and link this information to new cellular behaviors. One particular field in which gene-control elements evaluated via methods of the invention may find use includes the field of synthetic biology. Synthetic biology is a rapidly growing interdisciplinary field that involves the application of engineering principles to support the scalable construction and design of complex biological systems. One key focus of synthetic biology is to develop engineering frameworks for the reliable construction of genetic control devices that process and transmit information within living systems. Such information processing capabilities will allow researchers to implement diverse cellular control strategies, thus laying a critical foundation for designing genetic systems that exhibit sophisticated biological functions. The development of expression control systems, which may include evaluation of candidate gene-regulatory elements according to methods described herein, may find use in a variety of different fields, including but not limited to: health and medicine, environment, and sustainability. As such, embodiments of the invention find use in methods of generating gene regulatory components and devices with specific quantitative activities (e.g., by rapidly screening libraries of elements and obtaining higher enrichment efficiencies). Such generated regulatory elements can be used to precisely regulate gene expression. Methods of the invention also find use in screening for new sensing functions in cellular systems (developing ligand-responsive gene regulatory elements to new ligands).

### Computer Related Embodiments

Aspects of the invention further include a variety of computer-related embodiments. Specifically, the data analysis methods described in the previous sections may be performed using a computer. Accordingly, the invention provides a computer-based system for analyzing data produced using the above methods in order to evaluate a gene-regulatory element. In certain embodiments, the methods are coded onto a physical computer-readable medium in the form of “programming”, where the term “computer-readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. Of interest as media are non-transitory media, i.e., physical media in which the programming is associated with, such as recorded onto, a physical structure. Non-transitory media does not include electronic signals in transit via a wireless protocol. With respect to computer readable media, “permanent memory” refers to memory that is permanent. Permanent memory is not erased by termination of the electrical supply to a computer or processor. Computer hard-drive, CD-ROM, floppy disk and DVD are all examples of permanent memory. Random Access Memory (RAM) is an example of non-permanent memory. A file in permanent memory may be editable and re-writable.

### Kits

In yet another aspect, the present invention provides kits for practicing the subject methods, e.g., as described above. The subject kits may include a gene-regulatory element precursor, where the precursor is configured to be modified by a user to include a gene-regulatory element of interest. For example, the kit may include a nucleic acid construct, such as a plasmid, that includes a pro-activity reporter and a noise reporter. The pro-activity report may include a first signal reporter operatively coupled to a cloning site, e.g., a multiple cloning site, into which a user may readily insert a gene-regulatory element (or library of gene-regulatory elements) of interest. When the user inserts the gene-regulatory element of interest into the cloning site, the activity reporter then includes an expression cassette in which the first reporter domain is operatively coupled to the gene-regulatory element. In addition, the kits may include one or more additional compositions that are employed, including but not limited to: insertion reagents, e.g., restriction enzymes, phage mediated cloning reagents, cre-lox based cloning reagents, etc. (e.g., for use the insertion of a gene-regulatory element into the construct), buffers, diluents, host cells, etc., which may be employed in a given assay. The above components may be present in separate containers or one or more components may be combined into a single container, e.g., a glass or plastic vial.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### I. A High-Throughput, Quantitative Cell-Based Screen for Efficient Tailoring of RNA Device Activity

### A. Materials and Methods

### 1. Plasmid and Strain Construction

Standard molecular biology cloning techniques were used to construct all plasmids (Sambrook J RD (2001) Molecular Cloning: A Laboratory Manual, 3rd edn, Cold Spring Harbor, N.Y.: Cold Spring Harbor Lab Press). DNA synthesis was performed by Integrated DNA Technologies (Coralville, Iowa) or the Protein and Nucleic Acid Facility (Stanford, Calif.). All enzymes, including restriction enzymes and ligases, were obtained through New England Biolabs (Ipswich, Mass.). Ligation products were electroporated with a GenePulser XCell (Bio-Rad, Hercules, Calif.) into an E. coli DH10B strain (Invitrogen, Carlsbad, Calif.), where cells harboring cloned plasmids were maintained in Luria-Bertani media containing 50 mg/mL ampicillin (EMD Chemicals, Philadelphia, Pa.). All cloned constructs were sequence verified by Elim Biopharmaceuticals (Hayward, Calif.).

The two-color screening plasmid pCS1748 (FIG. 20) was constructed by inserting an open reading frame (ORF) encoding a yeast-optimized mCherry gene (ymCherry) flanked by a TEF1 promoter and a CYC1 terminator upstream of the existing ORF encoding a yeast-enhanced GFP gene (yEGFP3) flanked by a GAL1-10 promoter and an ADH1 terminator in the pCS321 backbone (Win & Smolke, “A modular and extensible RNA-based gene-regulatory platform for engineering cellular function,” Proc. Nat'l Acad. Sci. (2007) 104:14283-14288. The TEF1 promoter was PCR amplified from pG2M (Uppaluri & Towle, “Genetic dissection of thyroid hormone receptor beta: identification of mutations that separate hormone binding and transcriptional activation,” Mol. Cell. Biol. (1995) 15: 1499-1512) using forward and reverse primers SacI-TEF1-fwd (5′-GAGAGCTCAAGCTTCAAAATGTTTCTACTCC) (SEQ ID NO:01) and SacII-TEF1-rev (5′-GGCCGCGGCAAAACTTAGATTAGATTGCTATGC) (SEQ ID NO:02), respectively, and inserted into pCS321 via the unique restriction sites SacI and SaCII. The ymCherry gene was PCR amplified from BBa_E2060 obtained from the iGEM parts registry (Shaner et. al., “Improved monomeric red, orange and yellow fluorescent proteins derived from Discosoma sp. red fluorescent protein,” Nat. Biotechnol. (2004) 22: 1567-1572) using forward and reverse primers SacII-mCherry-fwd (5′-GACCGCGGGAAATAATGTCTATGGTTAGTAAAGGAGAAGAAAATAACATGG C) (SEQ ID NO:03) and NotI-mCherry-rev (5′-GGGCGGCCGCTTATTATTTGTATAGTTCA TCCATGCCACCAG) (SEQ ID NO:04), respectively, and inserted downstream of the cloned TEF1 promoter via the unique restriction sites SacII and NotI. The CYC1 terminator was PCR amplified from pCM159 (Gari et al, “A set of vectors with a tetracycline-regulatable promoter system for modulated gene expression in Saccharomyces cerevisiae,” Yeast (1997) 13: 837-848) using forward and reverse primers NotI-CYC1t-fwd (5′-GAGCGGCCGCGAGGGCCGCATCATGTAATTAG) (SEQ ID NO:05) and XbaI-CYC1t-rev (5′-GGTCTAGAGGCCGCAAATTAAAGCCTTCG) (SEQ ID NO:06), respectively, and inserted downstream of the cloned ymCherry gene via the unique restriction sites NotI and XbaI. A spacer sequence was amplified from pCS745 (gift from M. Jensen, Seattle Children's Research Institute) using forward and reverse primers XbaI-Spacer-fwd (5′-GGTCTAGACGCCTTGAGCCTGGCGAACAGTTC) (SEQ ID NO:07) and Spacer-rev (5′-AGTAAAAAAGGAGTAGAAACATTTTGAAGCTATCGAT GACAGGATGAGGATCGTTTCGCATG) (SEQ ID NO:08), respectively. A TEF1 promoter fragment was amplified from pG2M using forward and reverse primers Spacer-TEF1-fwd (5′-CATGCGAAACGATCCTCATCCTGTCATCGATAGCTTCAAATG TTTCTACTCCTTTTTTACT) (SEQ ID NO:09) and BamHI-TEF1-rev (5′-GGGGATCCCA AAACTTAGATTAGATTGCTATGCTTTCTTTC) (SEQ ID NO:10), respectively, and PCR assembled with the spacer fragment. The assembled spacer-TEF1 promoter fragment was inserted into the modified pCS321 backbone to replace the GAL1-10 promoter via the unique restriction sites XbaI and BamHI.

Two single-color plasmids harboring GFP (pCS1585) and mCherry (pCS1749) were constructed as compensation controls for FACS analysis. A fragment harboring the TEF1 promoter was PCR amplified from pGM2 using forward and reverse primers SacI-TEF1-fwd (5′-GAGAGCTCATAGCTTCAAAATGTTTCTACTCC) (SEQ ID NO:11) and EcoRI-TEF1-rev (5′-GGGAATTCTTTGTAATTAAAACTTAGATTAGA) (SEQ ID NO:12), respectively. The GFP-only plasmid pCS1585 was constructed by inserting the TEF1 promoter fragment into pCS321 to replace the GAL1-10 promoter via the unique cloning sites SacI and EcoRI. The mCherry-only plasmid pCS1749 was constructed by inserting the TEF1p-ymCherry-CYC1t cassette from pCS1748 into a modified version of pRS316 (Sikorski & Hieter, “A system of shuttle vectors and yeast host strains designed for efficient manipulation of DNA in Saccharomyces cerevisiae,” Genetics (1989) 122: 19-27) via the unique restriction sites SacI and XbaI. The modified version of pRS316 (pCS4), containing no fluorescence reporter gene, was used as the negative-control construct.

Ribozyme-based devices and appropriate controls were inserted into the 3′ untranslated region (UTR) of yEGFP3 in pCS1748 through appropriate restriction endonuclease and ligation-mediated cloning. DNA fragments encoding the ribozyme-based devices and controls were PCR amplified using forward and reverse primers L1-2-fwd (5′-GACCTAGGAAACAAACAAAGCTGTCACC) (SEQ ID NO:13) and L1-2-rev (5′-GGCTCGAGTTTTTATTTTTCTTTTTGCTGTTTCG) (SEQ ID NO:14), respectively, and inserted into pCS1748 via the unique restriction sites AvrII and XhoI, which are located 3 nts downstream of the yEGFP3 stop codon. Cloned plasmids were transformed into the budding yeast Saccharomyces cerevisiae strain W303α (MATα his3-11, 15 trp1-1 leu2-3 ura3-1 ade2-1) through a standard lithium acetate method (Gietz & Woods, “Transformation of yeast by lithium acetate/single-stranded carrier DNA/polyethylene glycol method,” Methods Enzymol. (2002) 350: 87-96). All yeast strains harboring cloned plasmids were maintained on synthetic complete media with a uracil dropout solution containing 2% dextrose (SC-URA) and grown at 30° C.

The mating fate routing plasmid pCS2293 was constructed by cloning a TEF1 mutant 7 (TEF1 m7) promoter (Nevoigt et al., “Engineering of promoter replacement cassettes for fine-tuning of gene expression in Saccharomyces cerevisiae,” Appl. Environ. Microbiol. (2006) 72: 5266-5273), and the coding region for Msg5 into the pCS321 backbone (Win & Smolke, 2007 supra). The TEF1m7 promoter was PCR amplified from pCS1142 using forward and reverse primers pTEF7-fwd (5′-AAGAGCTCATAGCTTCAAAATGTCTCTACTCCTTTTT) (SEQ ID NO:15) and pTEF7-rev (5′-AAAGGATCCAACTTAGATTAGATTGCTATGCTTTCTTTCC) (SEQ ID NO:16), respectively, and inserted into pCS321 via the unique restriction sites SacI and BamHI. The MSG5 gene was PCR amplified from genomic DNA using forward and reverse primers Msg5.K2-fwd (5′-AAAGGATCCAATTAATAGTGCACATGCAATTTCAC) (SEQ ID NO:17) and Msg5-rev (5′-AAAACCTAGGTTAAGGAAGAAACATCATCTG) (SEQ ID NO:18), respectively, and inserted via the unique restriction sites BamHI and AvrII. Ribozyme-based devices and appropriate controls were inserted into the 3′ UTR via the unique restriction sites AvrII and XhoI, located immediately downstream of the MSG5 stop codon as described previously.

The mating reporter construct (pCS1124) was built by cloning FUS1p, a mating-responsive promoter, into the pCS321 backbone (Win & Smolke, 2007, supra). The FUS1 promoter was PCR amplified from pDS71 (Siekhaus & Drubin, “Spontaneous receptor-independent heterotrimeric G-protein signalling in an RGS mutant,” Nat. Cell. Biol. (2003) 5:231-235) using forward and reverse primers pDS71.Fus1-fwd (5′-TTTGCGGCCGCCCAATCTCAGAGGCTGAGTCT) (SEQ ID NO:19) and pDS71.Fus1-rev (5′-TTTGGATCCTTTGATTTTCAGAAACTTGATGGC) (SEQ ID NO:20), respectively, and inserted upstream of yEGFP3 in pCS321 via the unique restriction sites NotI and BamHI. The FUS1p-yEGFP3-ADH1t cassette from pCS1124 was cloned into pCS1391, a loxP integrating vector (Guldener et al., “A new efficient gene disruption cassette for repeated use in budding yeast,” Nucleic Acids Res. (1996) 24: 2519-2524), via the unique restriction sites SacI and KpnI to make pCS2292. FUS1p-yEGFP3-ADH1t was chromosomally integrated into yeast strain EY1119 (W303a Δsst1 Δkss1::HIS3) (Flotho et. al., “Localized feedback phosphorylation of Step 5p scaffold by associated MAPK cascade,” J. Biol. Chem. (2004) 279: 47391-4740) via homologous recombination using the gene cassette from pCS2292 to construct yeast strain CSY840 (W303a Δsst1 Δkss1::HIS3 trp1::FUS1p-yEGFP3-ADH1t-loxP-KanR). Briefly, the FUS1p-yEGFP3-loxP-KanR cassette was PCR amplified using Expand High Fidelity PCR system (Roche, Indianapolis, Ind.) from pCS2292 using forward and reverse primers TRP1.INT.all.fwd (5′-GTATACGTGATTAAGCACACAAAGGCAGCTTGGAGTA TGTCTGTTATTAATTTCACAGGAAGATTGTACTGAGAGTGCAC) (SEQ ID NO:21) and TRP1.INT.all.rev (5′-TTGCTTTTCAAAAGGCCTGCAGGCAAGTGCACAAACAATA CTTAAATAAATACTACTCAGCGACTCACTATAGGGAGACC) (SEQ ID NO:22), respectively, each carrying 60 nts of homologous sequence to the TRP1 locus. Yeast strain EY1119 was transformed with 12 μg of gel purified PCR product and plated on G418 plates to build yeast strain CSY840. All plasmids and yeast strains constructed in this study are summarized in Table S1.

TABLE 1
Summary of plasmids and yeast strains constructed.

| Plasmid | Color Controls |
|---|---|
| pCS1748 | two-color screening plasmid: TEF1p-GFP-ADH1t |
| | and TEF1p-mCherry-CYC1t |
| pCS1585 | single-color plasmid: TEF1p-GFP-ADH1t |
| pCS1749 | single-color plasmid: TEF1p-mCherry-CYC1t |

Ribozyme-based Devices and Controls

| | |
|---|---|
| pCS1750 | pCS1748 + sTRSV |
| pCS1751 | pCS1748 + sTRSV Contl |
| pCS1752 | pCS1748 + L2b1 |
| pCS1753 | pCS1748 + L2b8 |
| pCS2260 | pCS1748 + L2b5 |
| pCS2261 | pCS1748 + L2b8-a1 |
| pCS2262 | pCS1748 + L2b8-a6 |
| pCS2263 | pCS1748 + L2b8-a9 |
| pCS2264 | pCS1748 + L2b8-a10 |
| pCS2265 | pCS1748 + L2b8-a11 |
| pCS2266 | pCS1748 + L2b8-a12 |
| pCS2267 | pCS1748 + L2b8-a14 |
| pCS2268 | pCS1748 + L2b8-a27 |
| pCS2269 | pCS1748 + L2b8-t11 |
| pCS2270 | pCS1748 + L2b8-t24 |
| pCS2271 | pCS1748 + L2b8-t47 |
| pCS2272 | pCS1748 + L2b8-t197 |
| pCS2273 | pCS1748 + L2b8-t241 |
| pCS2274 | pCS1748 + L2b8-a1-t41 |
| pCS2275 | pCS1748 + L2b8-a1-t55 |
| pCS2276 | pCS1748 + L2b8-a1-t64 |
| pCS2277 | pCS1748 + L2b1-a1 |
| pCS2278 | pCS1748 + L2b1-a14 |
| pCS2279 | pCS1748 + L2b5-a1 |
| pCS2280 | pCS1748 + L2b5-a14 |
| pCS2281 | pCS1748 + L2bOFF1 |
| pCS2282 | pCS1748 + L2bOFF1-a1 |
| pCS2283 | pCS1748 + L2bOFF1-a14 |
| pCS2284 | pCS1748 + L2b8tc |
| pCS2285 | pCS1748 + L2b8tc-a1 |
| pCS2286 | pCS1748 + L2b8tc-a14 |
| pCS2287 | pCS1748 + sTRSV-a1 |
| pCS2288 | pCS1748 + sTRSV-a14 |
| pCS2289 | pCS1748 + L2b8-a1-t11 |
| pCS2290 | pCS1748 + L2b8-a1-t47 |
| pCS2291 | pCS1748 + L2b8-t41 |

Yeast Mating Pathway

| | |
|---|---|
| pCS1124 | FUS1p-GFP-ADH1t |
| pCS2292 | FUS1p-GFP-ADH1t in LoxP integrating vector |
| pCS2293 | TEF1m7p-Msg5-ADH1t |
| pCS2294 | pCS2293 + sTRSV |
| pCS2295 | pCS2293 + L2b8-a1 |
| pCS2297 | pCS2293 + L2b8 |
| pCS2299 | pCS2293 + sTRSV Contl |

Strain

| | |
|---|---|
| CSY840 | W303a Δsst1 Δkss1::HIS3 trp1::FUS1p-yEGFP3- |
| | ADH1t-loxP-KanR |

### 2. Library-Scale Yeast Transformation

device libraries (FIG. 21) were amplified using forward and reverse primers gap-L1-2-fwd (5′-TCCATGGTATGGATGAATTGTACAAATAAAGCCTAGGAAACAAAC AAAGCTGTCACC) (SEQ ID NO:23) and GAP-L1-2-rev (5′-AAGAAATTCGCTTATTTA GAAGTGGCGCGCCTCTCGAGTTTTTATTTTTCTTTTTGCTGTTTCG) (SEQ ID NO:24), respectively. The library was inserted into pCS1748 through homologous recombination-mediated gap-repair during transformation into yeast strain W303 (Chao et. al., “Isolating and engineering human antibodies using yeast surface display,” Nat. Protoc. (2006) 1: 755-768). Briefly, an 800 μL library PCR reaction was performed with 160 pmol of each primer and 16 pmol of the library template. 8 μg of the plasmid pCS1748 was digested with AvrII and XhoI. The digested vector was combined with the library PCR product, extracted with phenol chloroform, and precipitated into a dry pellet with ethanol. A Tris-DTT buffer (2.5 M DTT, 1 M Tris, pH 8.0) was added to a 50 mL yeast culture (OD₆₀₀ 1.3-1.5) and incubated at 30° C. for 10-15 min. The yeast were pelleted, washed with chilled Buffer E (10 mM Tris, pH 7.5, 2 mM MgCl₂, 270 mM sucrose), and resuspended in Buffer E to a final volume of 300 μL. The yeast mixture was directly added to the precipitated DNA pellet and 504 of the mixture was transferred to a chilled 2 mm gap cuvette for electroporation (540 V, 25 μF, 1000Ω). Following transformation, the cells were resuspended in 1 mL warmed YPD media and incubated at 30° C. for 1 hr. Multiple transformations (˜5) were performed to cover the desired library diversity (˜10⁶-10⁷). Transformation efficiencies were determined by plating serial dilutions of the transformants, and transformed cells were propagated in SC-URA media for FACS.

### 3. Two-Color FACS-Based Screen

Cells harboring the RNA device libraries and control constructs were washed, resuspended in FACS buffer (1% BSA in PBS), and stained with a DAPI viability dye (Invitrogen). The cell suspension was filtered through a 40 μM cell strainer (BD Systems, San Jose, Calif.) prior to analysis on a FACSAria II cell sorter (BD Systems). GFP was excited at 488 nm and measured with a splitter of 505 nm and bandpass filter of 525/50 nm. mCherry was excited at 532 nm and measured with a splitter of 600 nm and a bandpass filter of 610/20 nm. DAPI was excited at 355 nm and measured with a bandpass filter of 450/50 nm. A scatter gate was set based on the forward and side-scatter area of cells harboring the negative-control plasmid (pCS4) to exclude debris, followed by a DAPI-(−) viability gate to exclude dead cells in the DAPI-(+) gate from the analysis. Cells harboring the single-color control plasmids (pCS1585, pCS1749) were analyzed to compensate spillover from GFP to the mCherry detector. A general sorting strategy was followed in which cells harboring devices with targeted activities were analyzed to set a sorting gate on a two-dimensional scatter plot that correlates GFP and mCherry fluorescence. Cells within this gate were collected into SC-URA media and propagated to sufficient density for further screening or analysis.

### 4. Ribozyme-Based Device Characterization Through Flow Cytometry Analysis

Enriched device libraries from FACS were directly plated onto SC-URA solid medium. Individual colonies were screened and characterized for gene-regulatory activity of the devices based on flow cytometry analysis. The GFP fluorescence was measured on a Quanta flow cytometer (Beckman Coulter, Fullerton, Calif.). GFP was excited at 488 nm and measured with a splitter of 488 nm and a bandpass filter of 525/40 nm. Cells harboring the negative-control plasmid (pCS4) were analyzed to set the GFP-(−) and GFP-(+) gate. Activities were determined as the geometric mean of the GFP fluorescence based on the GFP-(+) population using FlowJo (Tree Star), and normalized to the geometric mean of the GFP fluorescence of a positive control (sTRSV Contl, a noncleaving sTRSV ribozyme with a scrambled core) that is grown under identical ligand conditions, run in the same experiment, and set to 100%.

Devices that exhibited desired activities were amplified by colony PCR using forward and reverse primers CS653 (5′-GGTCACAAATTGGAATACAACTATAACTCT) (SEQ ID NO:25) and CS654 (5′-CGGAATTAACCCTCACTAAAGGG) (SEQ ID NO:26), respectively, and sequenced. The recovered devices were resynthesized and recloned into the vector backbone to confirm the observed activity. DNA oligos were synthesized and amplified for insertion into pCS1748 using forward and reverse primers L1-2-fwd (5′-GACCTAGGAAACAAACAAAGCTGTCACC) (SEQ ID NO:27) and L1-2-rev (5′-GG CTCGAGTTTTTATTTTTCTTTTTGCTGTTTCG) (SEQ ID NO:28), respectively. The resynthesized devices were inserted into pCS1748 via the unique restriction sites AvrII and XhoI. The reconstructed device plasmids were transformed into the W303 yeast strain through a standard lithium acetate method (Gietz & Woods, supra). Cells harboring the selected devices and appropriate controls were prepared as described above for the sorting experiments and analyzed on an LSRII flow cytometer (BD Systems) to characterize the gene-regulatory activity of each device. GFP was excited at 488 nm and measured with a splitter of 505 nm and a bandpass filter of 525/50. mCherry was excited at 532 nm and measured with a splitter of 600 nm LP and a bandpass filter of 610/20 nm. DAPI was excited at 405 nm and measured with a bandpass filter of 450/50 nm. FlowJo was used to process all flow cytometry data. Cells harboring pCS4 and pCS1749 were analyzed to set the mCherry-(−) and mCherry-(+) gates. Activities in the absence or presence of ligand were determined as the geometric mean of the GFP fluorescence based on the mCherry-(+) population, and normalized to the geometric mean of the GFP fluorescence of a positive control (sTRSV Contl, a noncleaving sTRSV ribozyme with a scrambled core) in the absence or presence of ligand, respectively, to correct for any non-specific effects of ligand on the measured fluorescence (FIG. 16). Reported device activities are the mean and standard deviation of at least three independent experiments.

### 5. In Vitro Transcription and Purification of Ribozyme-Based Devices

Selected ribozyme-based devices and ribozyme and noncleaving controls were PCR amplified to include an upstream T7 promoter site and spacer sequence and downstream spacer sequence using forward and reverse primers T7-L1-2-fwd (5′-TTCTAATACGACTCACTATAGGGACCTAGGAAACAAACAAAGCTGTCACC) (SEQ ID NO:29) and L1-2-rev (5′-GGCTCGAGTTTTTATTTTTCTTTTTGCTGTTTCG) (SEQ ID NO:30), respectively. A total of 1-2 μg of PCR product was transcribed in a 25 μl reaction, consisting of the following components: 1×RNA Pol Reaction Buffer (New England Biolabs), 2.5 mM rATP, 2.5 mM rCTP, 2.5 mM rUTP, 0.25 mM rGTP, 1 μl RNaseOUT (Invitrogen), 10 mM MgCl₂, 1 μl T7 Polymerase (New England Biolabs), and 0.5 μCi α-³²P-GTP (MP Biomedicals, Solon, Ohio). 400 pmol of antisense DNA oligonucleotide, device-blocking (5′-GGTGACAGCTTTGTTTGTTTCCTAGGTCCCCC) (SEQ ID NO:31) and sTRSV-blocking (5′-GCTGTTTCGTCCTCACG) (SEQ ID NO:32), was added to each reaction to inhibit cleavage of the RNA devices and sTRSV hammerhead ribozyme, respectively, during transcription. After incubation at 37° C. for 2 hr, NucAway Spin Columns (Ambion, Austin, Tex.) were used to remove unincorporated nucleotides from the transcription reactions according to manufacturer's instructions. 10 μl aliquots of the recovered RNA were mixed with 3 volumes of RNA stop/load buffer (95% formamide, 30 mM EDTA, 0.25% bromophenol blue, 0.25% xylene cyanol), heated at 95° C. for 5 min, snap cooled on ice for 5 min, and size-fractionated on a denaturing (8.3 M urea) 10% polyacrylamide gel at 25 W for 45 min. Gels were imaged by phosphorimaging analysis on a FX Molecular Imager (Bio-Rad). Uncleaved transcripts were gel extracted and recovered with the ZR Small-RNA™ PAGE Recovery Kit (Zymo Research, Irvine, Calif.) according to manufacturer's instructions. Samples were stored in sterile, nuclease-free, deionized water at −80° C. to limit the extent of RNA self-cleavage prior to performing the cleavage assays.

### 6. In Vitro Ribozyme Cleavage Assays

The purified uncleaved transcripts were incubated in 100 mM NaCl, 50 mM Tris-HCl (pH 7.5) at 95° C. for 5 min, cooled at a rate of −1.3° C. to 37° C., and held there for 10 min to allow equilibration of secondary structure. A zero time-point aliquot was taken prior to initiating the self-cleavage reaction at 37° C. with the addition of MgCl₂ to a final concentration of 500 μM. Reactions were quenched at specified time points with addition of 3 volumes of RNA stop/load buffer on ice. Samples were heated 95° C. for 5 min, snap cooled on ice for 5 min, and size-fractionated on a denaturing (8.3 M urea) 10% polyacrylamide gel at 25 W for 45 to 60 min. Gels were exposed overnight on a phosphor screen and analyzed for relative levels of the full-length transcript and cleaved products by phosphorimaging analysis. The cleaved product fraction at each time point (Fₜ) was fit to the single exponential equation F₁=F₀+(F_{∞}−F₀)×(1−e^{-kt}) using Prism 5 (GraphPad), where F₀ and F_{∞} are the fractions cleaved before the start of the reaction and at the reaction endpoint, respectively, and k is the first-order rate constant of self-cleavage. Reported values are the mean of at least three independent experiments.

### 7. Measuring Mating Pathway Activity Via a Transcriptional Reporter and Halo Assays

The mating fate routing plasmids with ribozyme-based devices and appropriate controls were transformed into yeast strain CSY840. Cells were inoculated into the appropriate drop-out media, grown overnight at 30° C., and back diluted into fresh media in the presence or absence of 5 mM theophylline to an OD₆₀₀ of <0.1. After growing for 3 hr at 30° C., cells were stimulated with saturating pheromone levels, to a final concentration of 100 nM α mating factor acetate salt (Sigma-Aldrich, St. Louis, Mo.), to activate the mating pathway. After 3 hr of stimulation, GFP fluorescence levels from the pFus1-yEGFP3 reporter were evaluated via flow cytometry using a Cell Lab Quanta SC flow cytometer (Beckman Coulter, Fullerton, Calif.). Normalized pathway activity is calculated as the geometric mean of three biological replicates of each sample normalized to the blank plasmid control (no MSG5) in the absence of theophylline. Mating associated cell-cycle arrest was evaluated via halo assays (Sprague, “Assay of yeast mating reaction,” Methods Enzymol. (1991) 194: 77-93). Halo assays were performed on cultures grown overnight, back diluted into fresh media in the absence of theophylline, and grown another 6 hr. 200 μl of each replicate was plated on the appropriate drop-out plates in the absence or presence of 5 mM theophylline. A gradient of a mating factor was established by saturating a filter disk (2 mm diameter) of Whatman paper with 9 μl of 0.1 mg/mL α mating factor and placing the disk on the center of the plate. Cells were grown for 18 hr at 30° C. and imaged via epi-white illumination with a GelDoc XR+ System (Bio-Rad). To control for any possible differences in growth rate between replicates, plates are compared within one biological replicate ensuring differences in growth rate arise from divergence following plating in the absence or presence of theophylline.

### B. Results

1. A two-color reporter construct supports a high-efficiency, high-throughput, Quantitative, Cell-Based Screening Strategy

Genetic control devices with desired regulatory activities are often generated through cell-based screening strategies by coupling the regulatory function to a measurable output. However, the efficiencies of high-throughput cell-based screening strategies based on measuring activities in single cells are negatively impacted by gene expression noise that arises from both extrinsic (e.g., cell size and shape, cell cycle stage, plasmid copy number) and intrinsic (e.g., fluctuation in numbers of DNA, RNA, transcription factors, environmental stimuli) factors (Elowitz et al., “Stochastic gene expression in a single cell,” Science (2002) 297: 1183-1186; Raser & O'Shea,

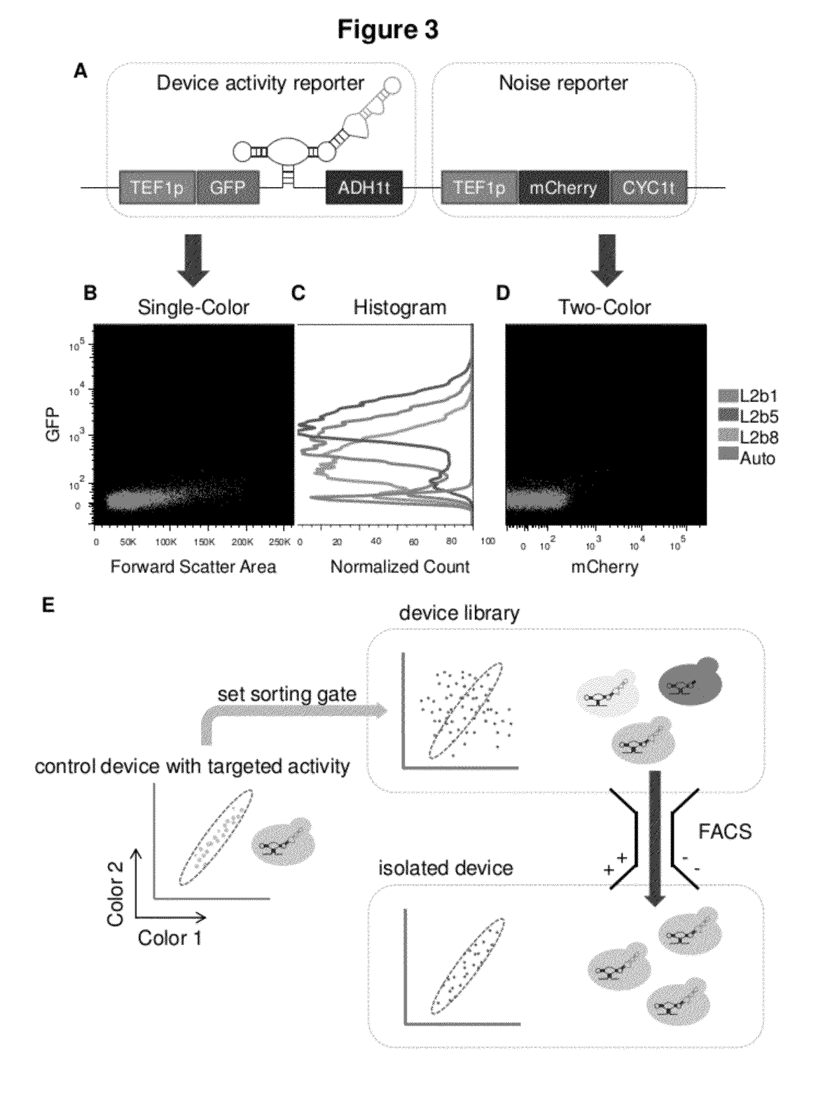

Control of stochasticity in eukaryotic gene expression,” Science (2004) 304: 1811-1814). To develop a more effective and quantitative in vivo screening strategy for gene-regulatory devices, we constructed a screening plasmid composed of two independent functional modules (FIG. 3A). The first module, the device activity reporter, utilizes a GFP reporter to measure the gene expression activity associated with the regulatory device, whereas the second module, the noise reporter, utilizes a mCherry reporter to provide a measure of cellular gene expression activity that is independent of the regulatory device. We used the same constitutive promoter (TEF1) in each module, although other promoter pairs may be substituted into the construct as previous studies in yeast suggest that extrinsic noise, which is the predominant source of gene expression noise, is not promoter specific (Raser & O'Shea, supra). Different terminators for each module were used to decrease the frequency of homologous recombination between the two modules.

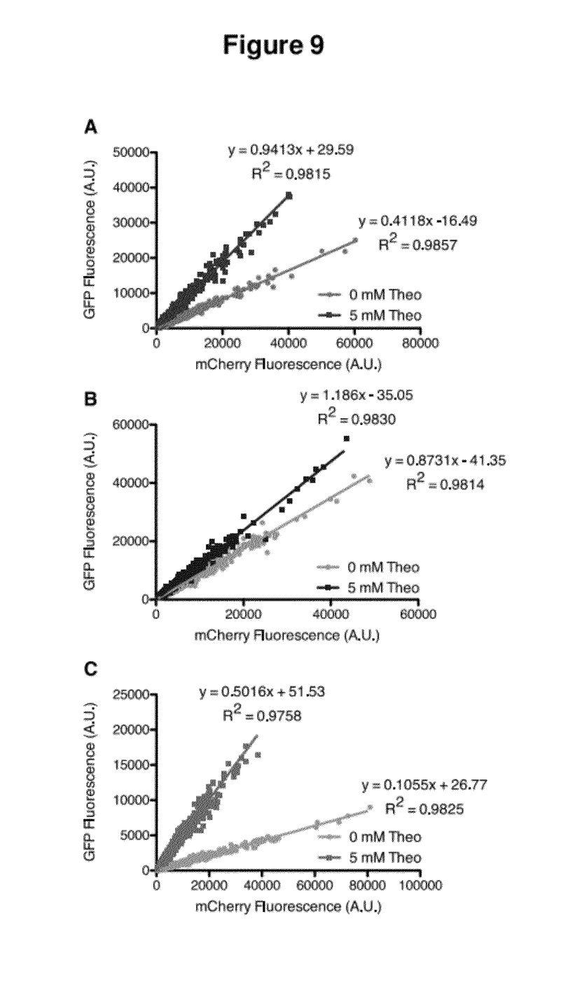

We first used the two-color screening construct to examine the basal activities of three previously characterized theophylline-responsive ribozyme-based devices (Win & Smolke (2007), supra) based on the output from the device activity reporter (GFP): L2bulge1 (L2b1), 40%; L2bulge5 (L2b5), 70%; and L2bulge8 (L2b8), 11%. The reported basal activities refer to the GFP expression levels relative to that of the inactive ribozyme control in the absence of theophylline. While these three devices exhibit a wide range of regulatory activities, cellular noise results in a broad distribution of GFP fluorescence levels, making it challenging to cleanly isolate members with target activities from a library through a FACS-based sorting strategy based solely on one output (FIG. 3B, C). To provide a filter for changes in gene expression activity due to cellular noise, we used the noise reporter in the two-color screening construct to assess activity that is independent of the regulatory device. By using the output from the noise reporter (mCherry), we were able to normalize the gene expression activities of individual cells by correlating the outputs between the two reporters and cleanly resolve the cell populations harboring three different devices (FIG. 3D). For each device, the cell population exhibits a tight linear relationship between the two outputs, such that the device regulatory activity is associated with a distinct slope (FIG. 9). The increased resolution between cell populations harboring devices with different regulatory activities enables the development of a high-efficiency, quantitative, two-color FACS-based screening strategy based on the output correlation between the two reporter modules (FIG. 3E).

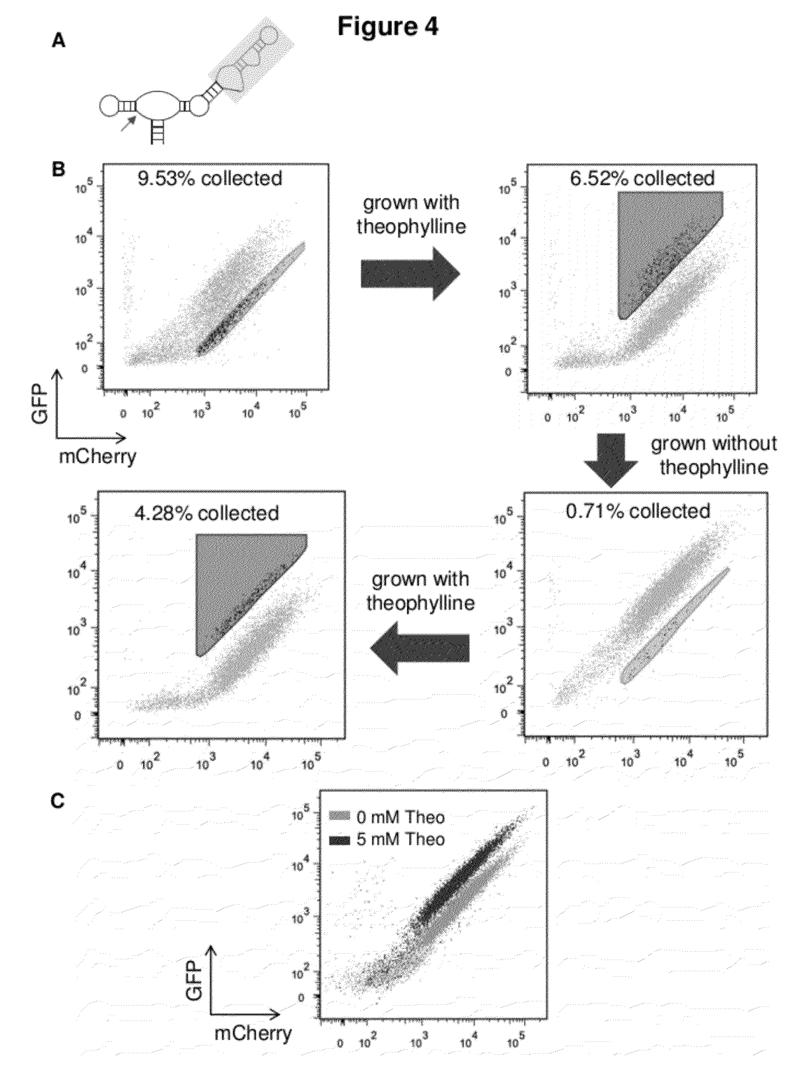

We demonstrated the high enrichment efficiency of our FACS-based two-color screening strategy by performing a control screen on a sensor library (sN10). The sN10 sensor library was generated by randomizing 10 nt positions in the theophylline aptamer binding core (sensor component) within the L2b8 device (˜1×10⁶ variants) (FIG. 4A). We first assessed the basal activity of the sN10 sensor library through two-color flow cytometry analysis and observed that the majority of the library exhibits higher activity (greater slope) than the parent device (FIG. 4B). We set a sorting gate based on the basal activity of the parent device (negative gate) and performed a negative sort to enrich cells (˜10% of the initial library) that exhibit similar basal activities (same slope). We calculated the fold enrichment based on the percentage of the cell population isolated from each sort. In the initial negative sort, we collected 9.53% of the cell population and therefore this population was enriched ˜11 times (100/9.53, or ˜11-fold) for the subsequent screen. We performed a positive sort on the recovered cells, in which the lower bound of the sorting gate was set by the activity of the L2b8 device in the presence of 5 mM theophylline. The positive sorting gate was expanded further to enrich members of the library (˜7% of the enriched library) that exhibit equal or increased activities in response to theophylline relative to the parent device by ˜15-fold. After one sorting cycle, a distinct small sub-population of cells that exhibit comparable basal activity to the parent device was observed. We then performed another round of sorting to specifically enrich this sub-population (˜1% of the enriched library) through a negative sort by ˜143-fold, followed by a positive sort on the recovered cells to enrich a clearly distinct cell population that exhibits increased gene expression activity (˜4% of the enriched library) in response to theophylline by ˜25-fold. After two sorting cycles, ˜80% of the enriched library exhibits a clear population shift in response to theophylline (FIG. 4C).

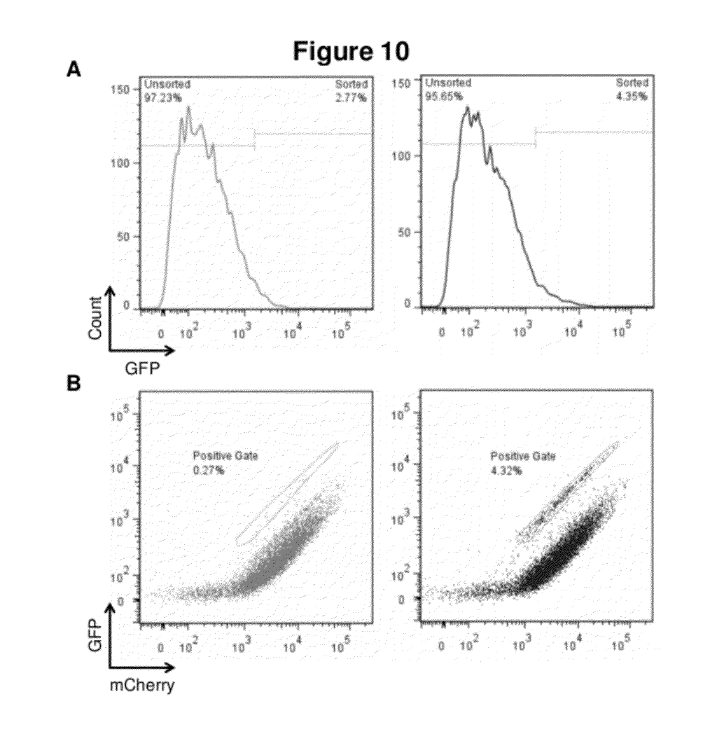

Since the two rounds of sorting yield an overall ˜6×10⁶-fold enrichment, given an initial library diversity of ˜10⁶ variants we expected to recover the original parent device from the enriched library by screening a small number of individual clones. We characterized 48 individual colonies from the enriched library for increased theophylline-responsive activities through flow cytometry. All 48 colonies exhibit similar increases in GFP expression levels in response to theophylline as the parent L2b8 device. We sequenced the recovered devices from 10 colonies, and all were verified to be the parent sequence. These results demonstrate the high efficiency of our two-color FACS-based screening strategy, which can enrich a single sequence from a large ˜10⁶ library to close to pure isolation in as few as two sorting rounds. The efficiency of our screening strategy is a direct result of the high resolving power of our two-color screening construct. For example, when examining the enriched library after one round of sorting, we could observe a distinct cell population that exhibits comparable basal activity to the parent L2b8 device on a two-color scatter plot (FIG. 10). However, when examining the cell population on a single-color (GFP) histogram, noise associated with the gene expression activity results in poor resolution between the device regulatory activity and cellular noise, thereby significantly decreasing the enrichment efficiency of a screening strategy based on a single output.

2. Screening of an Actuator Library Results in RNA Control Devices with Improved Regulatory Stringencies

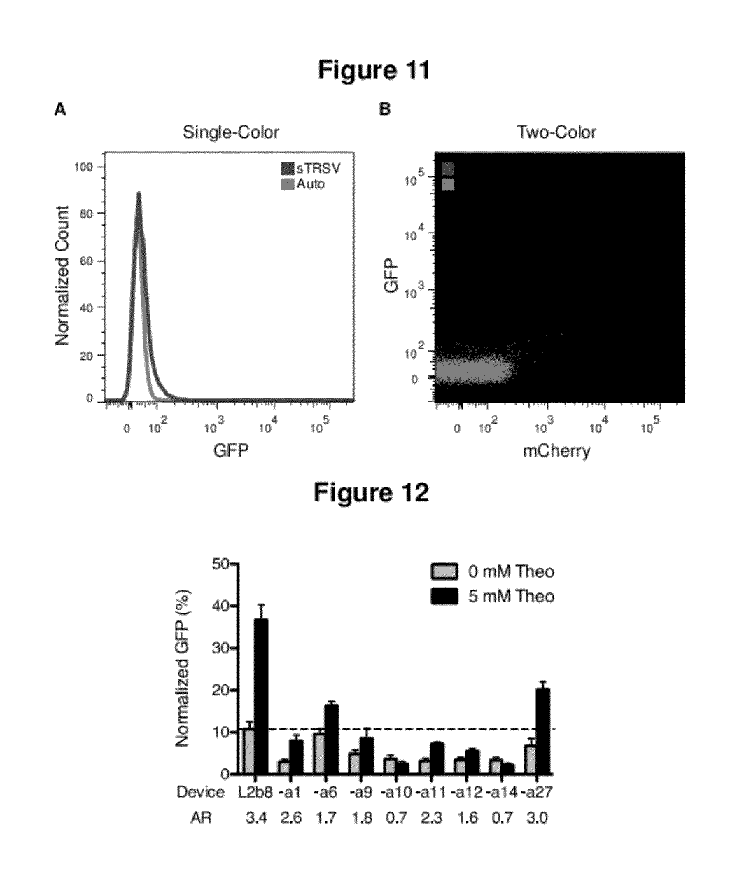

The implementation of RNA control devices in biological systems requires flexible tailoring of regulatory functions, where regulatory stringency of a device can be an important property for crossing phenotypic thresholds (Chen et al., “Genetic control of mammalian T-cell proliferation with synthetic RNA regulatory systems,” Proc. Nat'l Acad. Sci. USA (2010) 107: 8531-8536). The basal activity of a single ribozyme-based device depends on both the actuator and transmitter components. The catalytic activity of the hammerhead ribozyme (actuator) within the context of the device platform sets a lower limit to the minimal gene expression level a device exhibits in the absence of ligand, whereas the transmitter directs the partitioning between the functional conformations, which in turn impacts the basal activity (Win & Smolke, 2007, supra). The device platform specifies the integration of a sensor-transmitter element through loop II (or loop I) of the satellite tobacco ringspot virus (sTRSV) ribozyme actuator. Previous sTRSV ribozyme characterization studies have demonstrated that a tertiary interaction between loop I and loop II is essential for the catalytic activity of the ribozyme in cellular environments (Khvorova et al., “Sequence elements outside the hammerhead ribozyme catalytic core enable intracellular activity,” Nat. Struct. Biol. (2003) 10: 708-712). The integration of additional structural elements through the ribozyme loops may negatively impact the required loop-loop interaction, thus resulting in a less active ribozyme within the device platform. The previously described theophylline-responsive ribozyme devices (L2b1, 40%; L2b5, 70%; L2b8, 11%) exhibit a basal expression activity higher than that exhibited by the ribozyme alone (1%) (FIG. 11), suggesting that the current natural ribozyme sequence in the device platform may limit our ability to generate RNA control devices that exhibit tighter regulatory stringencies.

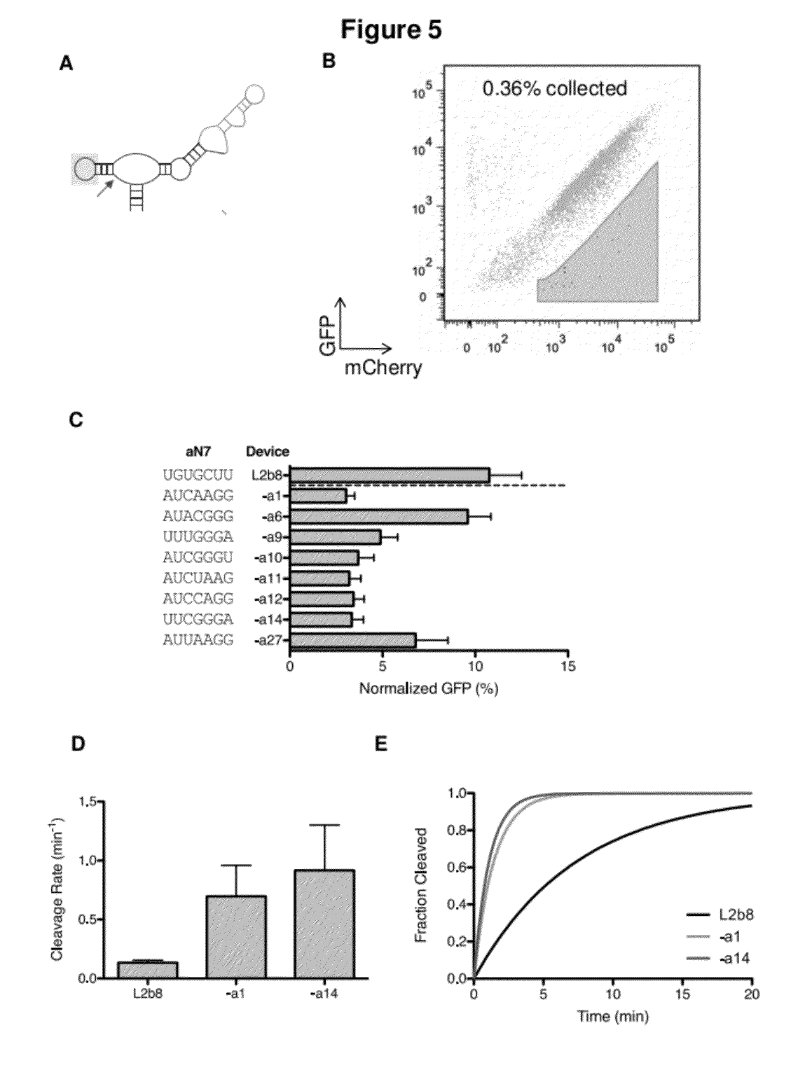

We focused initially on optimizing the sTRSV ribozyme sequence in the L2b8 device as it has the lowest basal activity among the series of previously engineered theophylline-responsive devices (Win & Smolke, 2007, supra). As prior sTRSV ribozyme characterization studies have demonstrated that mutations to either loop I or II sequences can enhance or hinder ribozyme cleavage activity (Khvorova, supra), we designed a device library by randomizing loop sequences and applied our two-color FACS-based screening approach to search for improved loop-loop interactions that support lower basal activity. We generated an actuator library (aN7) by randomizing 7 nts in loop I within the L2b8 device (˜2×10⁴ variants) (FIG. 5A). Loop I was targeted for randomization as it is isolated from the conformational change that is facilitated by the transmitter component, which is integrated via loop II. We analyzed the initial aN7 actuator library by two-color flow cytometry analysis and observed that ˜99% of the library exhibits a higher basal activity (greater slope) than the parent L2b8 device, indicating that the majority of loop I library sequences have deleterious effects on ribozyme catalytic activity (FIG. 5B). The two-color approach also enables us to clearly distinguish cells with low gene expression activity from cells that have lost or mutated the plasmid, whereas the two cell populations are almost indistinguishable if GFP is used as the only measure of device regulatory activity (FIG. 11). We set a sorting gate based on cells harboring the parent L2b8 device and collected cells that exhibit a decreased slope relative to the parent device (˜0.4% of the initial library) to enrich library members with enhanced loop-loop interactions that support lower device basal activity.

Due to the relatively small sequence space of the library and high-efficiency of our screen, we directly plated the sorted cells to obtain single colonies for further characterization. We recovered a total of 22 clones and identified 22 unique device sequences from the recovered clones. However, only eight of the recovered devices maintained low basal activities upon re-cloning (FIG. 5C), while the remaining devices likely had mutations within the recovered plasmid backbone. The basal activities exhibited by the recovered devices are up to 3.5-fold lower than that of the parent device, while five of the devices retained functional switching (i.e., responsiveness to ligand) (FIG. 12).

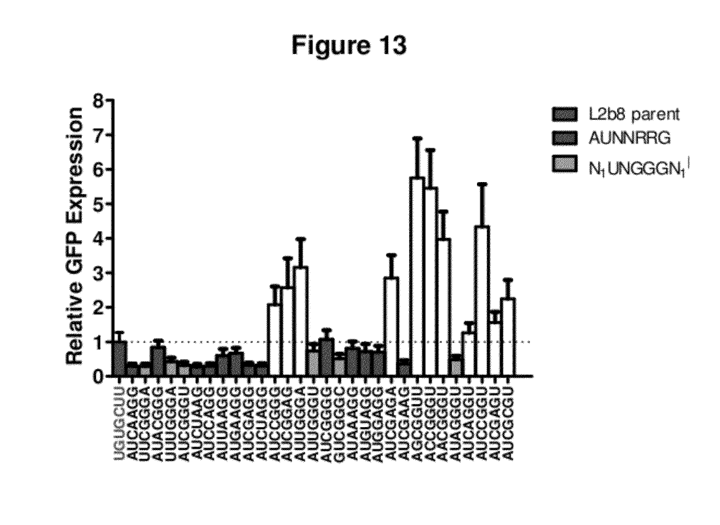

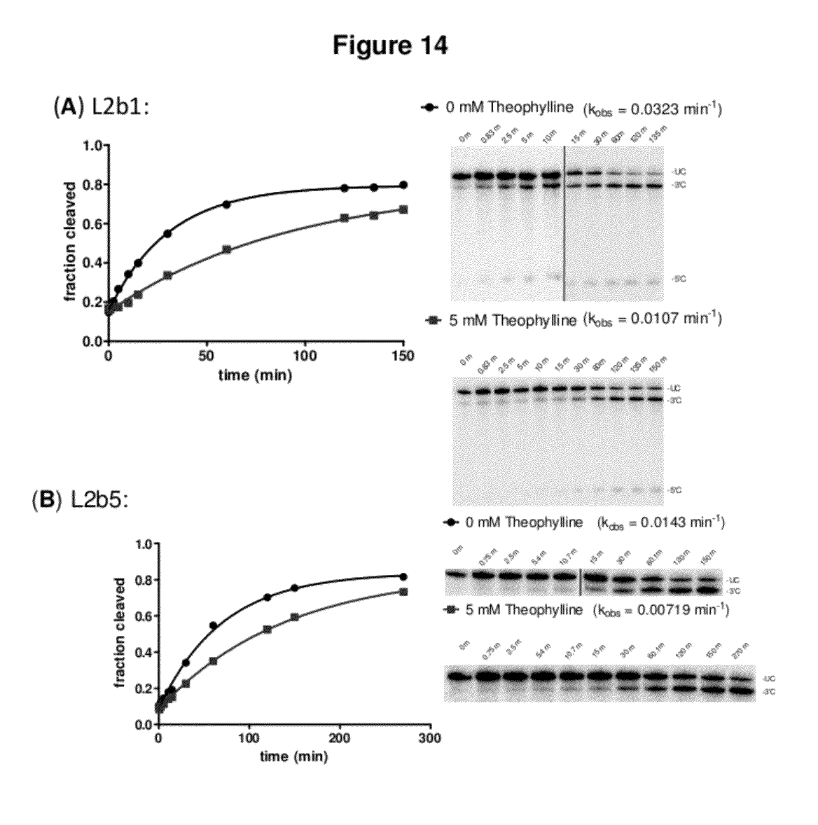

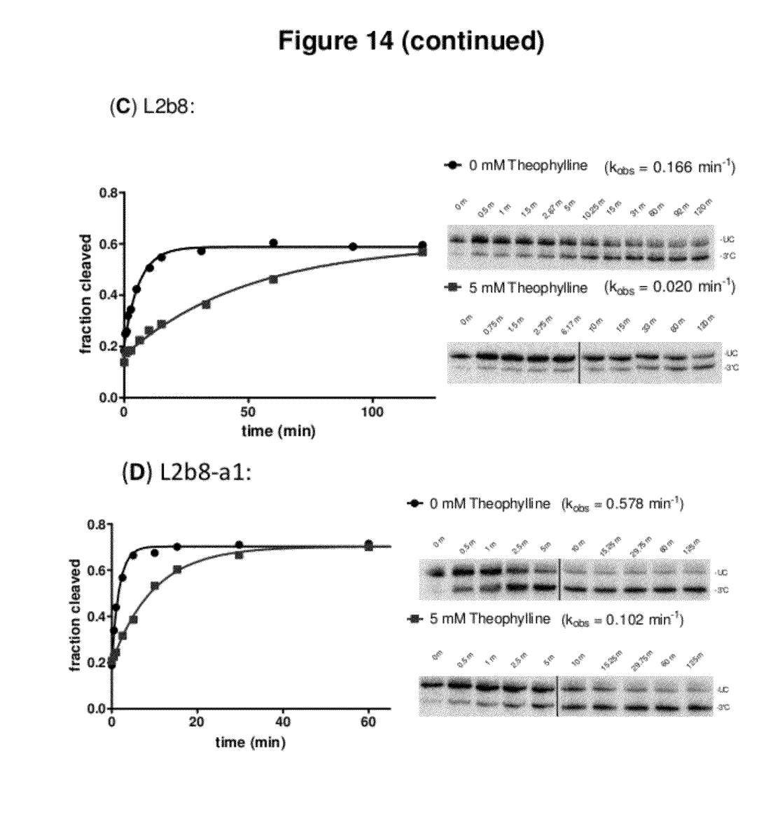

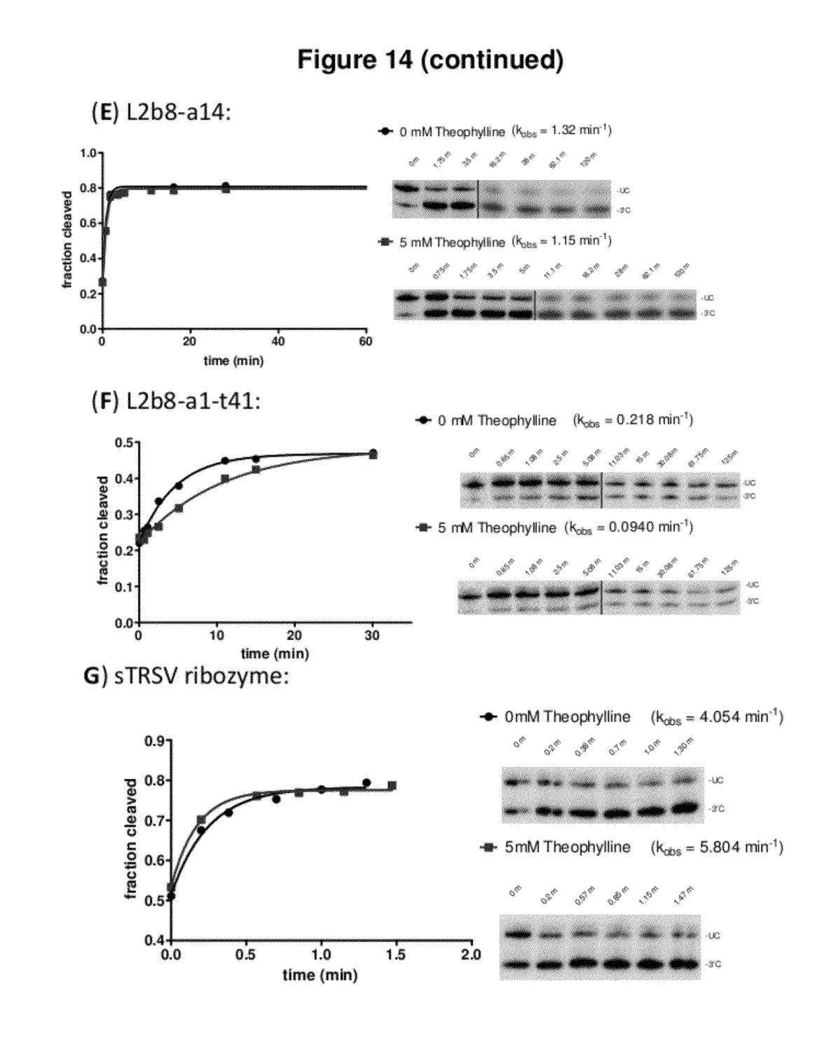

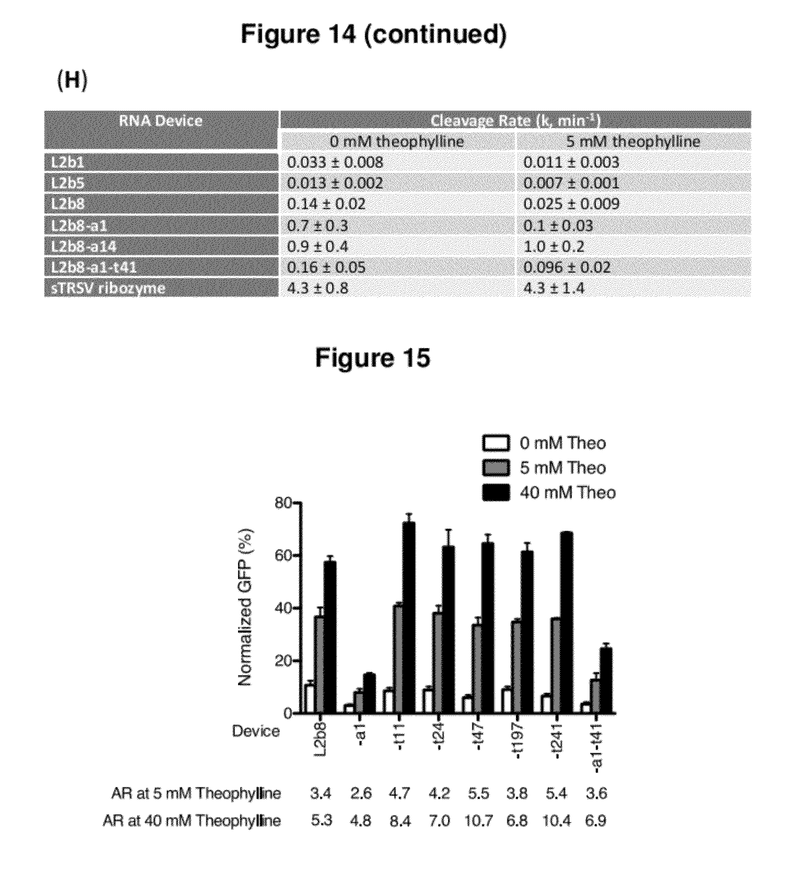

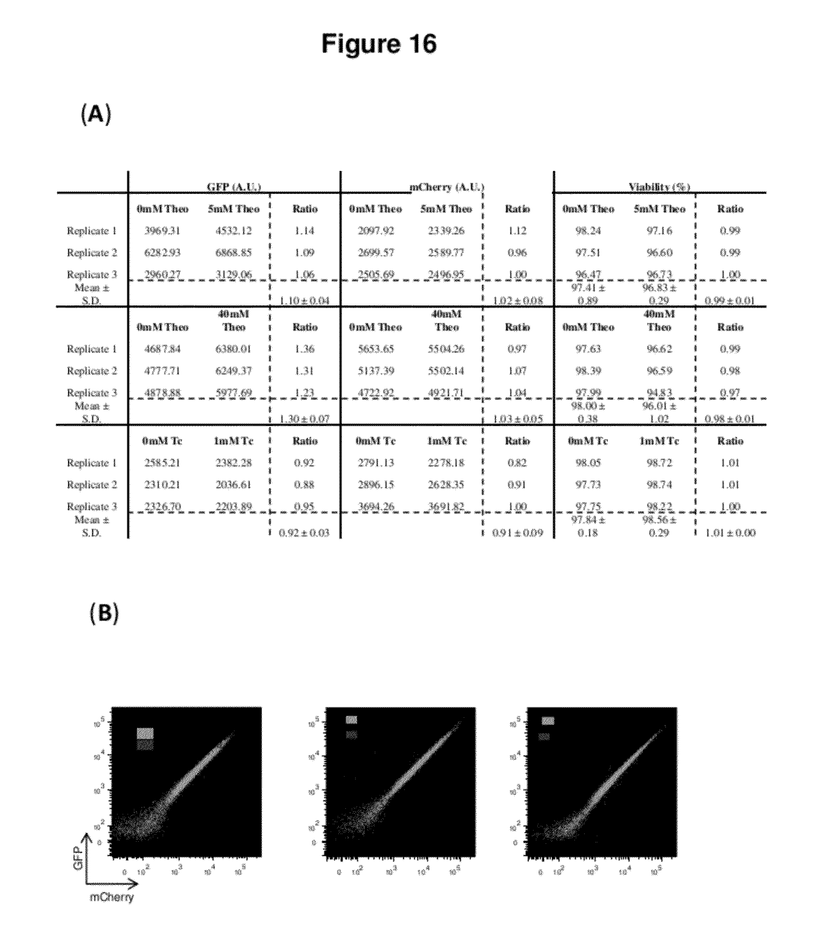

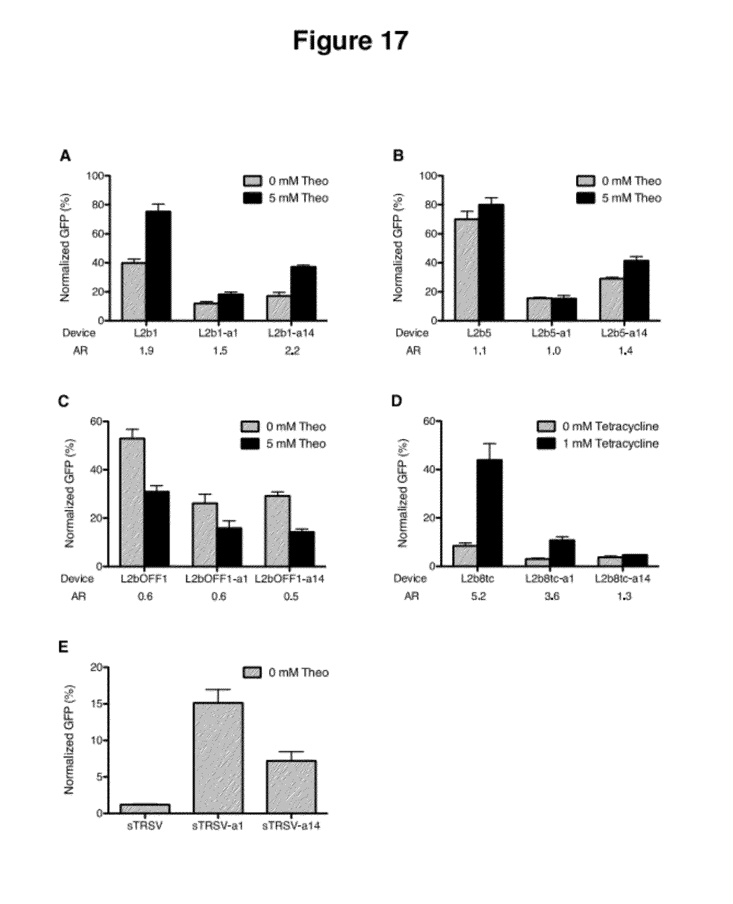

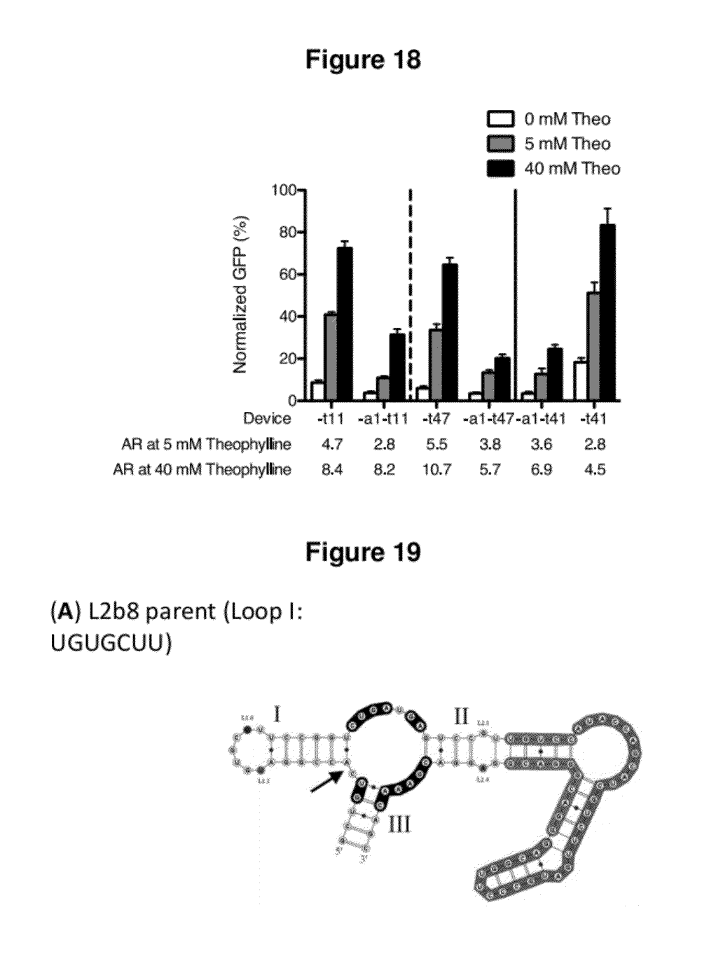

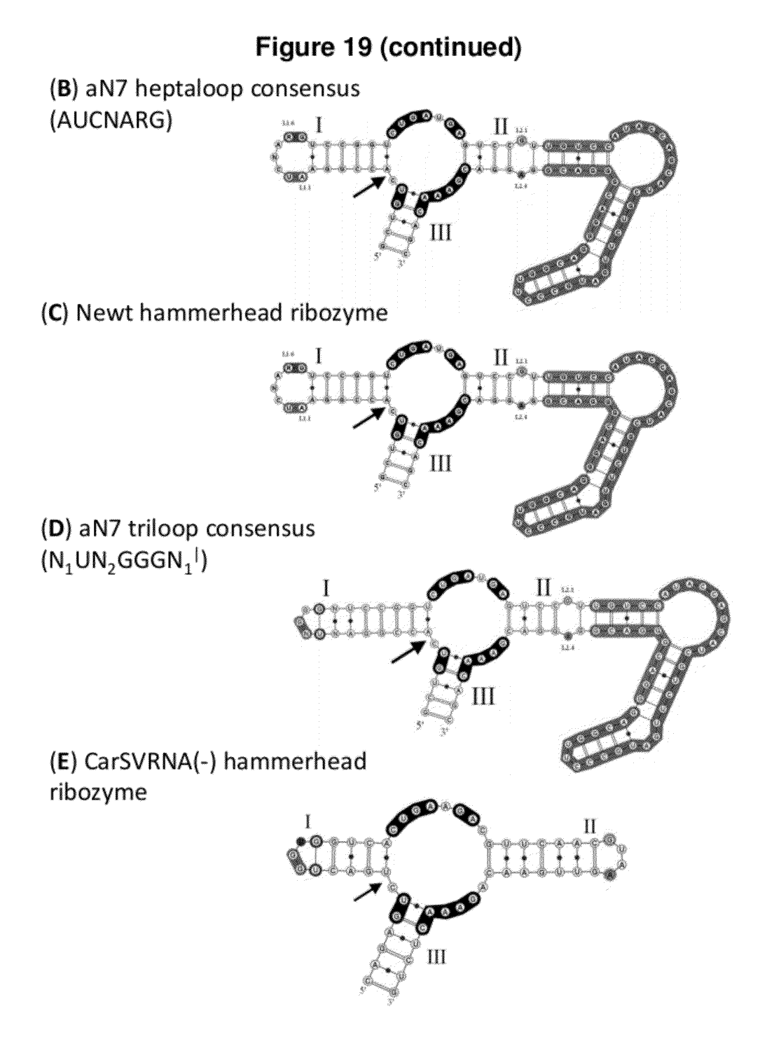

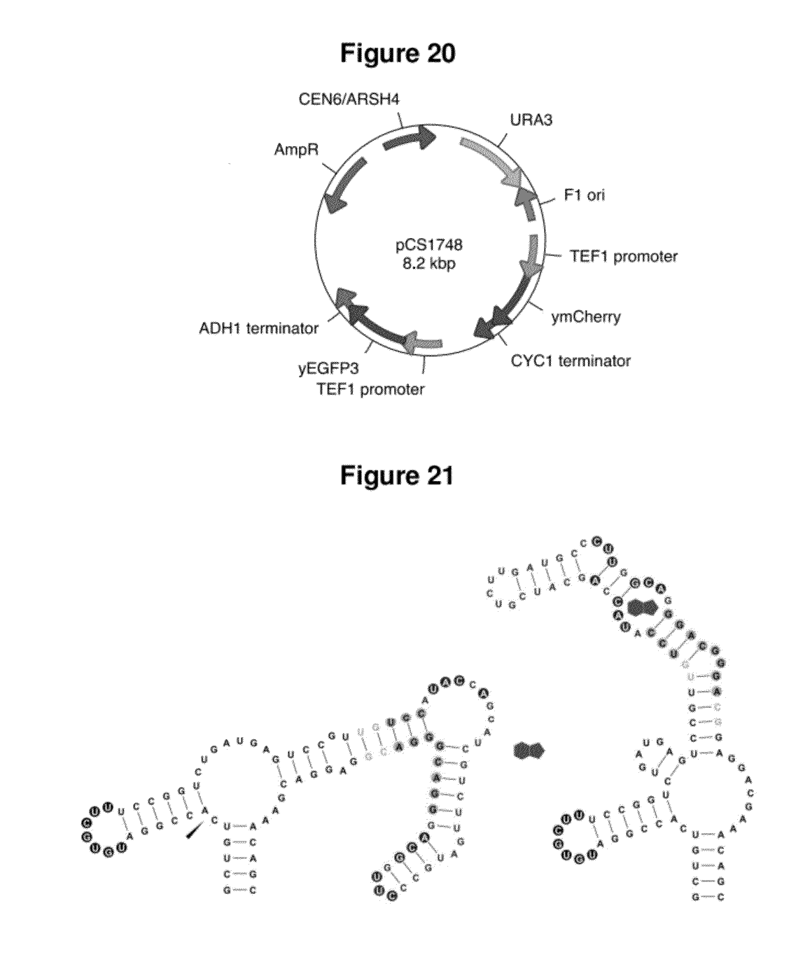

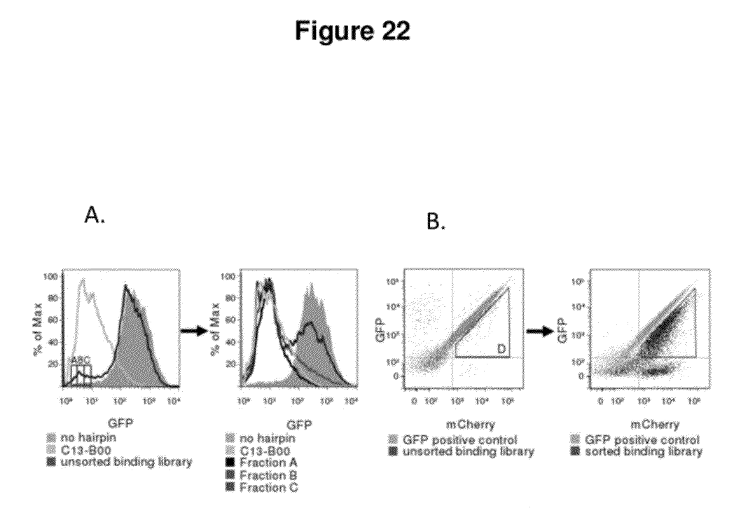

We performed a sequence analysis to identify the loop I sequence requirements that support high ribozyme catalytic activity within the device platform. A series of point mutations to the recovered loop I sequences were designed, and the activities of these loop I sequences were characterized through flow cytometry analysis (FIG. 13). Two distinct consensus sequences were identified that support lower basal activities than the parent L2b8 device. RNA devices with a loop I heptaloop adhering to the consensus sequence AUNNRRG, where N is any nucleotide base and R is a purine base, exhibit basal activities that are less than or equal to that of the parent (11%). A subset of these devices with the loop I consensus sequence AU(C/U/G)NARG exhibit basal activities less than 80% of that of the L2b8 parent device. Further restricting the loop I consensus sequence to AUCNARG results in additional improvements to the basal activity, to 40% of that of the parent. RNA devices with a second, distinct loop I consensus sequence predicted to form a triloop N₁UN₂GGGN₁^{I}, where N₁ and N₁^{I} are any Watson-Crick base pair, exhibit improved basal activities less than 70% of that of the L2b8 parent.

### 3. Engineered Actuator Components Result in Optimized Ribozyme-Based Devices Exhibiting Faster Cleavage Rates

In vitro kinetic analysis of select ribozyme-based devices harboring loop I sequence modifications was performed to assess whether the improved in vivo basal activities were a result of increased catalytic rate relative to the parent device. Cleavage rates (k) were determined at physiologically-relevant reaction conditions (500 μM MgCl₂, 100 mM NaCl, and 50 mM Tris-HCl (pH 7.0)) at 37° C., where the submillimolar magnesium concentration is within the range estimated for intracellular magnesium concentration (London, “Methods for measurement of intracellular magnesium: NMR and fluorescence,” Annu. Rev. Physiol. (1991) 53: 241-258). Cleavage rate constants were obtained for the L2b8 parent device and the L2b8-a1 and -a14 engineered devices (FIG. 5D, E) by quantifying relative levels of full-length and cleaved radiolabeled transcripts through polyacrylamide gel electrophoresis analysis (FIG. 14). Compared to the cleavage rate constant of L2b8 (0.14 min⁻¹), the cleavage rates associated with the engineered devices are increased 5- and 6.8-fold for L2b8-a1 (0.70 min⁻¹) and -a14 (0.92 min⁻¹), respectively. The cleavage rate of the sTRSV hammerhead ribozyme (4.3 min⁻¹) was determined at these conditions to be 45-fold greater than L2b8 (FIG. 14), in agreement with prior analyses on this ribozyme (Khvorova et al., supra) and other well characterized natural ribozymes under physiologically relevant conditions (Canny et al., “Fast cleavage kinetics of a natural hammerhead ribozyme,” J. Am. Chem. Soc. (2004) 126: 10848-10849). The results indicate that the modified loop I sequences produce ribozyme-based devices exhibiting faster cleavage rates and thus lower basal expression levels.

### 4. Screening of Transmitter Libraries Results in RNA Devices Exhibiting Improved Activation Ratios

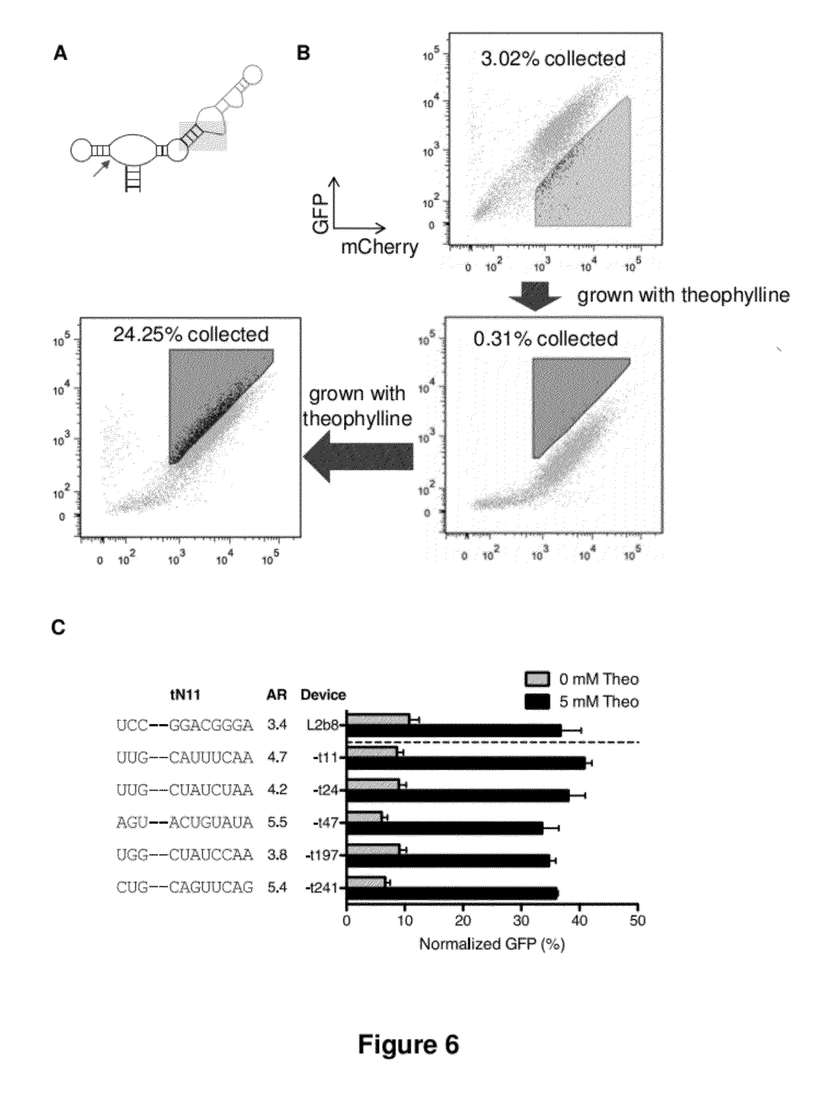

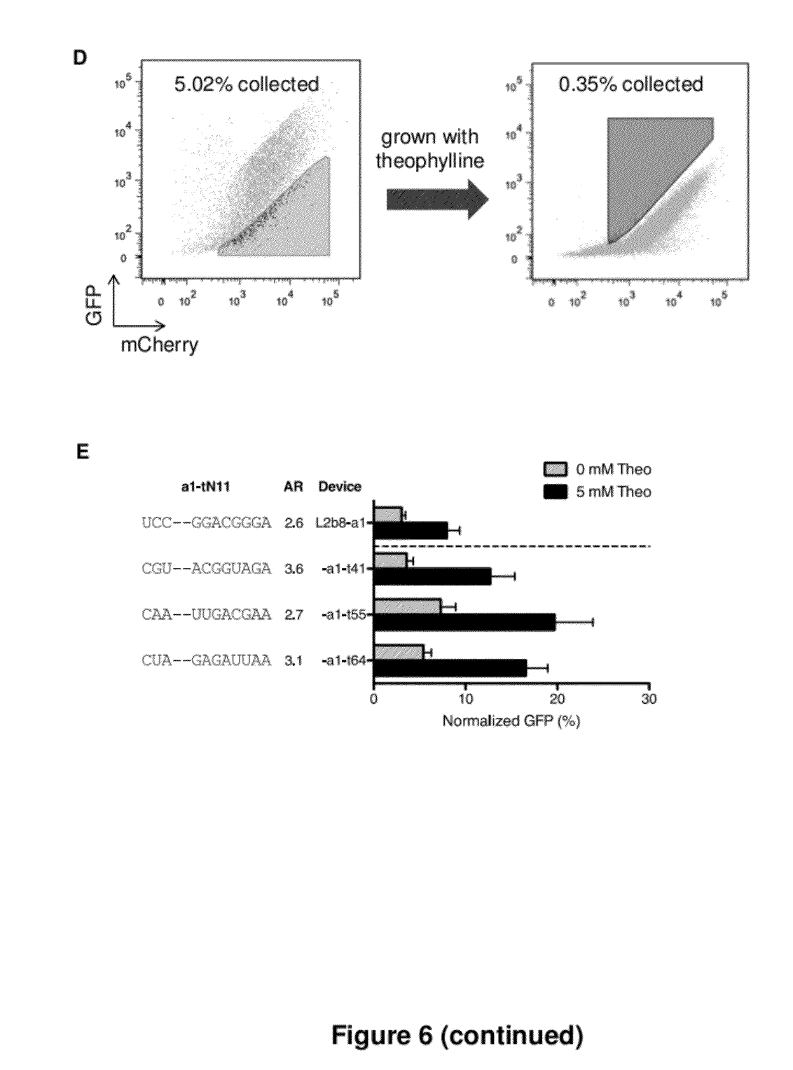

The dynamic range of an RNA control device depends on many parameters, including irreversible rate (e.g., ribozyme cleavage activity), intracellular ligand concentration, and mechanism by which binding information at the sensor is transmitted to an activity change in the actuator (e.g., transmitter design). We applied our two-color FACS-based screening strategy to explore a greater functional space to search for new transmitter sequences that support improved activation ratios given the designated sensor and actuator component pairs. We generated two transmitter libraries, tN11 and a1-tN11, based on two theophylline-responsive ribozyme-based devices, L2b8 and L2b8-a1, respectively, with ribozyme components that exhibit varying catalytic activities. Both libraries were generated by randomizing 11 nts in the transmitter component within the corresponding parent device (˜4×10⁶ variants) (FIG. 6A). We limited the number of randomized nucleotides in the transmitter components to result in libraries that could be completely searched based on the efficiency of our yeast library transformation method (see Materials and Methods). Both libraries were screened to identify devices with comparable basal activities but with greater activation ratios relative to the corresponding parent devices (FIG. 6B, D). We first performed a negative sort on both libraries by setting the sorting gates based on the basal activity of the respective parent device. The negative sort enriched members of the library (˜3% of the initial tN11 library; ˜5% of the initial a1-tN11 library) that exhibit lower or comparable basal activities as the parent device. A subsequent positive sort was performed on the recovered cell populations by setting sorting gates based on the gene expression activity of the respective parent device in the presence of 5 mM theophylline. To enrich members of the library that exhibit improved activation ratios, cells that exhibit equal or higher gene expression activities (˜0.3% of the enriched tN11 library; ˜0.4% of the enriched a1-tN11 library) were isolated. One additional positive sort was performed on the enriched tN11 library to further enrich for cells that exhibit activation ratios greater than or equal to the parent device (˜24% of the enriched library).

We plated the recovered cells from both final enriched libraries and screened 287 and 207 individual colonies from the tN11 and a1-tN11 libraries, respectively, through flow cytometry in the absence and presence of 5 mM theophylline and verified improved device activation ratios by re-cloning. We identified a total of five (t11, t24, t47, t197, t241) and three (a1-t41, a1-t55, a1-t64) transmitter variants from the tN11 and a1-tN11 library screen, respectively, that exhibit moderately improved activation ratios up to 160% relative to the parent devices (FIG. 6C, E). We speculated that the intracellular theophylline concentration in yeast might limit the maximum activation ratio achievable by a device, as previous studies have estimated an 1,000-fold drop in theophylline concentration across the Escherichia coli cell membrane (Koch & Lamont, 1956). We tested this hypothesis by characterizing the activation ratios of the transmitter variants at a higher theophylline concentration (40 mM) and observed devices with activation ratios up to 10.7-fold, corresponding to up to a 290% improvement in activation ratios relative to 5 mM theophylline (FIGS. 15, 18). No difference in cell viability was observed under all theophylline conditions tested (FIG. 16A), although the higher theophylline concentration resulted in slower cell growth, possibly due to the ligand cytotoxicity, thus potentially rendering the implementation of these devices at higher ligand concentrations less effective for cellular applications.

### 5. Modular Assembly of Optimized Actuator Components Results in Devices Exhibiting Improved Regulatory Stringencies

The modular composition framework of our ribozyme device platform supports modular assembly of functional RNA components to generate new device functions without significant redesign (Win & Smolke, 2007, supra; Win & Smolke, “Higher-order cellular information processing with synthetic RNA devices,” Science (2008) 322: 456-460. We examined the ability of the optimized ribozyme sequences to be used to build new device regulatory functions, by coupling two of the recovered ribozyme variants (a1, a14) with different sensor and transmitter components. We coupled the new actuator sequences with the transmitter and sensor components from two theophylline-responsive ribozyme ON devices (L2b1, L2b5), one theophylline-responsive OFF device (L2bOFF1), and one tetracycline-responsive ON device (L2b8tc) to generate eight new devices. All newly generated devices exhibit lower basal activities relative to their respective parent, and the majority of them retain switching activity (FIGS. 17A-D). The ribozyme variants were also coupled with selected transmitter variants to generate new functional devices with varying regulatory stringencies and activation ratios (FIG. 18). The results indicate that the improved ribozyme variants are relatively insensitive to the sequence identities of the other device components (transmitter, sensor) and highlight that the modular device platform can be used in combination with improved components to program RNA devices exhibiting desired activities. Finally, we examined the activities of the loop sequences outside of the device context by replacing the wild-type loop I sequence of sTRSV to generate two ribozyme variants (FIG. 17E). The gene expression activities of the resulting ribozyme variants were substantially higher than that of wild-type sTRSV, indicating that the improvement in regulatory stringencies associated with these loop sequences were specific to the context of the device platform, thus supporting the importance of optimizing component functions directly within the device platform.

### 6. In Vitro Cleavage Kinetics Reflect In Vivo RNA Device Gene-Regulatory Activities

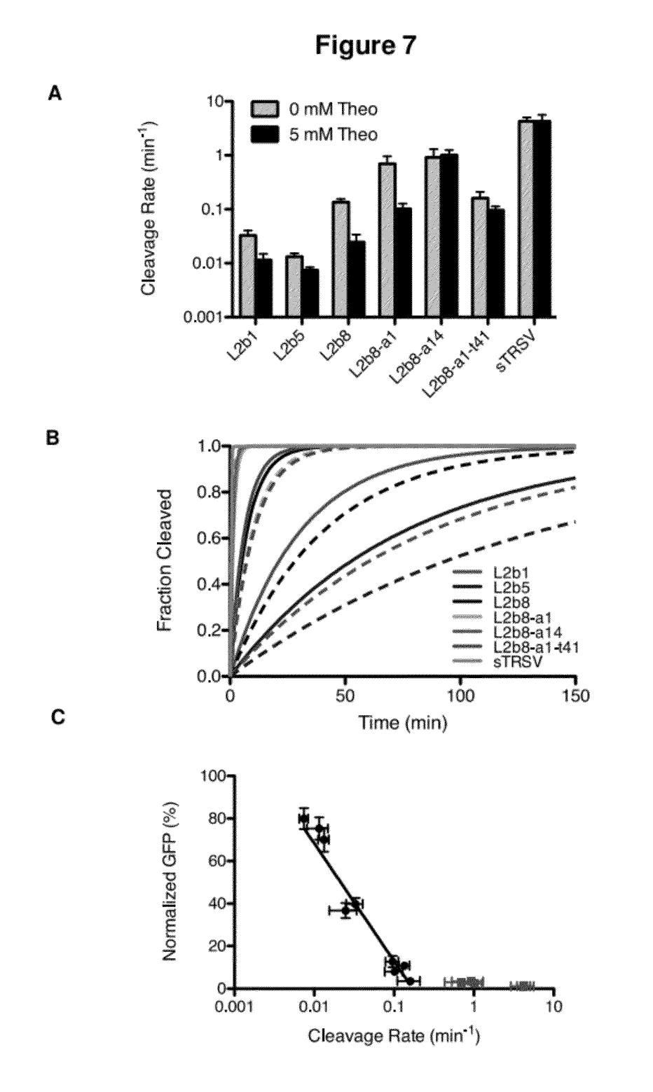

The observation that loop I modified ribozyme-based devices exhibit improved in vivo basal activities and in vitro cleavage rates relative to the L2b8 parent device suggests a correlation between these two measures of activity. However, previous studies had shown that allosteric ribozymes exhibiting enhanced in vitro cleavage rates through in vitro selections failed to exhibit enhanced in vivo gene-regulatory activities (Link et al., “Engineering high-speed allosteric hammerhead ribozymes,” Biol. Chem. (2007) 388: 779-786). Therefore, we examined the relationship between in vitro cleavage rates and in vivo gene-regulatory activities for a set of RNA devices generated through the two-color screening and rational tuning strategies. Cleavage assays were performed under the previously described physiologically-relevant reaction conditions in the absence and presence of 5 mM theophylline (FIG. 14). The ribozyme-based devices exhibit cleavage rates that span approximately two orders of magnitude from 0.01 min⁻¹ (L2b5) to 1.0 min⁻¹ (L2b8-a1, a14) (FIG. 7A, B). Nearly all of the examined RNA devices exhibit reduced cleavage rates in the presence of theophylline, except for L2b8-a14, which did not exhibit changes in gene-regulatory activity in response to theophylline (FIG. 12). The rank ordering of the examined RNA devices and the sTRSV ribozyme control by in vitro-determined cleavage rates matches the inverse ranking by in vivo gene-regulatory activities (FIG. 7C). A correlation analysis between these two measures of activity indicates a strong negative correlation (Pearson coefficient, r=−0.9018) between the cleavage rates 0.007 and 0.16 min⁻¹, where at a cleavage rate of 0.16 min⁻¹ near background levels of gene expression are reached such that further decreases in gene-regulatory activity are not observed with faster cleavage rates.

7. RNA Control Devices with Improved Regulatory Stringency Allow for Robust Redirecting of Yeast Mating Fates

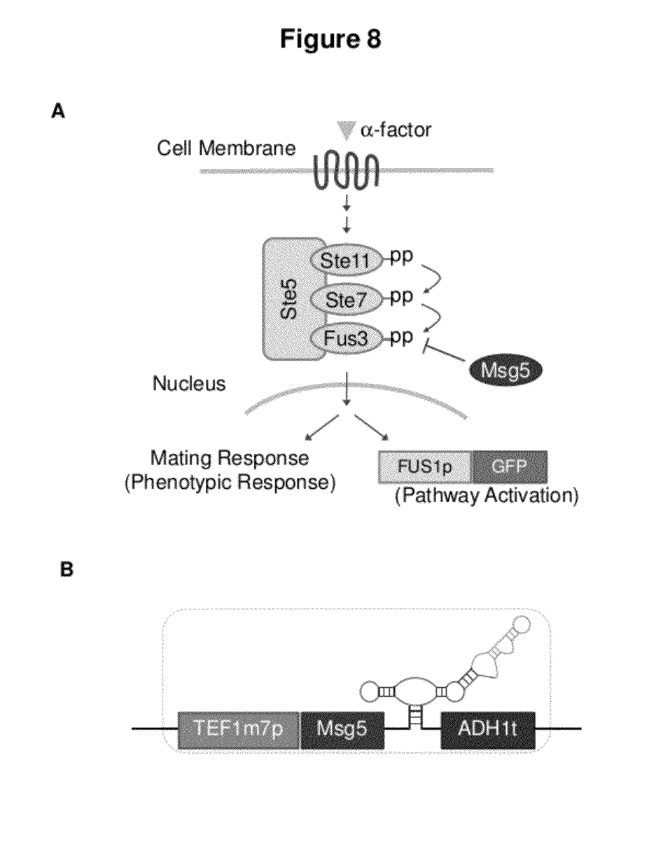

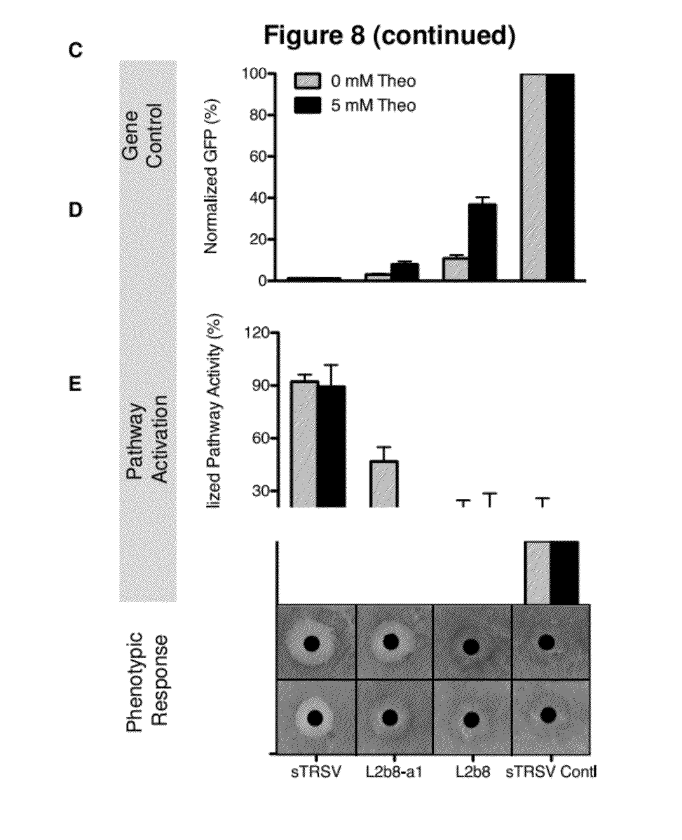

To achieve robust control of cell fate, the dynamic range of a genetic device must cross the threshold activity of the gene associated with the phenotypic change in the absence and presence of input molecule. RNA control devices have been integrated with biological systems to program complex cellular behaviors, such as proliferation and apoptosis (Chen, 2010, supra; Culler, “Reprogramming cellular behavior with RNA controllers responsive to endogenous proteins,” Science (2010) 330: 1251-1255). In these and other systems, devices with more stringent regulatory activities are necessary to sufficiently reduce background expression to achieve the desired control over the targeted phenotype. From our two-color FACS-based screens, we isolated new device variants that exhibit more than 3.5-fold lower basal activities relative to the parent device. To demonstrate the utility of devices with improved regulatory stringency, we applied these devices to control cell fate determination through a MAPK pathway associated with the mating response in yeast (FIG. 8A).

We targeted a key negative regulator in the mating pathway, Msg5, which is a MAPK phosphatase that inactivates signaling through this pathway by dephosphorylating the MAPK Fus3 (Andersson et al., “Differential input by Ste5 scaffold and Msg5 phosphatase route a MAPK cascade to multiple outcomes,” EMBO J. (2004) 23: 2564-2576). We first examined the impact on cell fate by ectopically expressing Msg5 regulated by either the wild-type ribozyme (sTRSV) or the inactive ribozyme (sTRSV Contl) controls (FIG. 8B, C). These two controls represent the minimal and maximal expression levels of Msg5 in our synthetic system, and the corresponding pathway activation was analyzed through a fluorescent reporter assay that measures transcriptional activation of the pathway and a phenotypic assay that measures cell cycle arrest in the presence of pheromone (FIG. 8D, E). The low expression level of Msg5 associated with the sTRSV construct allowed pathway activation in the presence of the pheromone, thereby leading to high GFP levels and wild-type halo formation. In contrast, the high expression level of Msg5 associated with the sTRSV Contl construct inhibited pheromone-induced pathway activation, thereby eliminating cell cycle arrest and resulting in low GFP levels.

To redirect yeast mating behavior in response to an exogenously applied molecular input, we implemented two theophylline-responsive ribozyme ON devices, L2b8 and L2b8-a1, to conditionally regulate expression of Msg5 (FIG. 8B, C). The L2b8 device exhibits the lowest basal expression activity (11%) among the devices previously engineered through a rational design strategy (Win & Smolke, 2007, supra). However, the basal expression level of Msg5 associated with the L2b8 device inhibited pathway signaling sufficiently to eliminate cell cycle arrest in the presence of pheromone even in the absence of theophylline (FIG. 8D, E). In contrast, the newly selected L2b8-a1 device exhibits a more stringent control profile than that of the L2b8 device and when applied to the regulation of Msg5 achieved rerouting of cell fate in response to the small molecule input. In the absence of theophylline, the basal expression level of Msg5 associated with the L2b8-a1 device was sufficiently low to permit elevated pathway activity in response to pheromone and characteristic halo formation as is observed for the wild-type ribozyme control. In the presence of theophylline, the increased expression level of Msg5 inhibited pathway activation sufficiently to reduce pheromone-induced cell cycle arrest, diminishing the appearance of the halo as is observed for the inactive ribozyme control. These results highlight the importance of tunable controllers, including those exhibiting stringent profiles, in targeting thresholds of key pathway components to achieve desired phenotypic switching. Although the L2b8 device exhibits a greater activation ratio relative to that of the L2b8-a1 device (FIG. 12), the higher basal activity of the L2b8 device rendered it ineffective in redirecting cell fate in this system.

### C. Discussion

We developed a two-color FACS-based screening approach that enables the efficient isolation of devices with targeted regulatory activities (enriching a single sequence from a large ˜10⁶ library to close to pure isolation in two sorting rounds), providing a substantial improvement over the enrichment efficiencies of more commonly used single output screening strategies that can be negatively impacted by noise associated with gene expression. We applied our two-color screening approach to optimize the actuator and transmitter components directly within the context of the ribozyme device platform in the relevant cellular environment, thereby addressing challenges previously encountered with optimizing component activities through in vitro strategies (Link et al., supra). The highly quantitative nature of our screening approach allowed us to efficiently isolate ribozyme and transmitter variants that exhibit specified regulatory activities. Our two-color screening approach is also fully complemented by the modular composition framework of the ribozyme-based platform. We demonstrated a plug-and-play device construction strategy by linking components with varying activities to generate new RNA devices that span wide regulatory ranges. In comparison to the previously demonstrated computational-guided device tuning strategy, which is restricted to experimentally sampling a smaller sequence space, our two-color screening approach offers greater flexibility and higher-throughput in quantitatively tailoring device regulatory properties.

We further characterized the isolated ribozyme and transmitter variants in addition to the devices generated previously by rational design through both in vivo and in vitro assays and found a strong negative correlation between gene-regulatory activities and cleavage rates, for cleavage rates within the range of 0.007 and 0.16 min⁻¹ (measured at the specified reaction condition of 500 μM MgCl₂). Gene expression levels reach background values at −0.16 min⁻¹, such that that increases in cleavage rate above this value have minimal impact on expression levels. In addition, our analysis (FIG. 6C) indicates that below cleavage rates of ˜0.007 min⁻¹ gene expression levels reach saturating (fully ON) levels, such that further decreases in cleavage rate below this value do not significantly impact gene expression. Our work demonstrates that there is a narrow window of in vitro cleavage rates (spanning less than two orders of magnitude) that correspond to titratable in vivo gene-regulatory activities. This observation may explain previous work in the field that observed no changes in gene-regulatory activities between ribozyme variants exhibiting different cleavage rates, if the changes in cleavage rates were occurring outside of the titratable window (Chen et al., “Direct selection for ribozyme cleavage activity in cells,” RNA (2009)15: 2035-2045).

Our results also highlight the importance of optimizing individual components within the context of device platforms. The parent actuator component (sTRSV ribozyme) is a type-III hammerhead ribozyme, in which the 5′ and 3′ ends of viroid RNA extend from stem III. The tertiary interactions essential for cleavage activity at physiological conditions are formed by a Watson-Crick base triple between the 5′ U of loop I, next-to-last U of loop I, and the 3′ A of loop II. Recent structural analyses have shown that these essential tertiary interactions are conserved across a significant fraction of natural hammerhead ribozymes (Dufour et al., “Structure-function analysis of the ribozymes of chrysanthemum chlorotic mottle viroid: a loop-loop interaction motif conserved in most natural hammerheads,” Nucleic Acids Res. (2009) 37: 368-381). As the loop I sequence of the originally designed ribozyme-based devices, including the parent device L2b8, is unchanged from the native sTRSV ribozyme (UGUGCUU), it was assumed that aptamer integration into loop II would maintain the same tertiary interactions required for cleavage activity (Win & Smolke, 2007, supra). However, both the heptaloop (AUCNARG) and triloop (N₁UN₂GGGN₁^{I}) consensus sequences identified through the actuator library screen that result in devices exhibiting lower basal activities than the L2b8 parent device do not follow the common tertiary motif of the sTRSV ribozyme. The divergence of the improved loop I sequences from the L2b8 and sTRSV parents are not necessarily surprising, given recent work identifying thousands of new hammerhead ribozymes in all domains of life with unique tertiary motifs (Dufour et al., supra; Perreault et al., “Identification of hammerhead ribozymes in all domains of life reveals novel structural variations,” PLoS Comput Biol (2011) 7: e1002031; Seehafer et al., “From alpaca to zebrafish: hammerhead ribozymes wherever you look,” RNA (2011) 17: 21-26). In particular, the integration of the transmitter-aptamer element into loop II of the sTRSV hammerhead ribozyme in the device platform makes the overall structure of the ribozyme-based devices more similar to other, less well-characterized hammerhead ribozymes (FIG. 19). Interestingly, the improved loop I consensus sequences identified in our screen share similarities to these other natural ribozymes and do not exhibit improved activities in the context of the sTRSV ribozyme alone.

Finally, we demonstrated the utility of our newly selected device variants to control the expression level of a key negative regulator in the yeast mating pathway through an exogenously applied molecular input. In this particular system, the regulated pathway component exhibits a low threshold level, thus requiring a regulatory device with low background activity to permit pathway activation in the absence of the effector molecule. We applied one of the selected device variants with improved regulatory stringency to achieve phenotypic switching in response to the exogenous molecular input, whereas the original device was ineffective in redirecting cell fate. These results highlight the importance of fine-tuning regulatory properties of gene-regulatory elements for their successful integration within biological systems. Recent examples have relied on using low-throughput and inefficient single-color screening strategies on individual clones from libraries of control elements, including promoters and RNase cleavage sites, to identify new elements that exhibit varying activities in different organisms (Alper et al., “Tuning genetic control through promoter engineering,” Proc. Nat'l Acad. Sci. USA (2005)102: 12678-12683; Babiskin & Smolke, “A synthetic library of RNA control modules for predictable tuning of gene expression in yeast,” Mol. Syst. Biol. (2011) 7: 471; Nevoigt et al., supra). In contrast, our two-color FACS-based screening approach is based on the correlation between the gene outputs from two independent functional modules. Similar dual-reporter screening plasmid systems can be constructed in other organisms, including bacteria and mammalian cells, to support the efficient generation of new gene control elements, including those acting through transcriptional and posttranscriptional mechanisms. Our strategy offers a high-throughput and high-efficiency alternative to rapidly screen through diverse libraries of control elements for members exhibiting specified quantitative activities, thereby addressing a key challenge in quantitatively tailoring these control elements to specific genetic systems.

### II. A High-Throughput, Quantitative Cell-Based Screen for Efficient Tailoring of RNase Element Activity

We extended the FACS-based strategy for the screening of an RNA component library that mediates the processing activity of an RNase III enzyme, Rnt1p, to identify a set of Rnt1p hairpins that will provide a range of different regulatory activities and be useful as modular gene-regulatory elements. The component library was generated by introducing 8 mutations in the protein-binding region of a functional RNA hairpin. Two different FACS-based screens were performed on the library. One screen was based on the single-color system, where cells that exhibit low fluorescence level were collected into three fractions (FIG. 22A). The other screen was based on the described two-color system, where cells that exhibit low fluorescence level were collected (FIG. 22B). Each of the three fractions collected from the single-color screen was regrown and characterized by flow cytometry. Only one of the three fractions retained a low level of fluorescence, whereas the other two fractions shifted toward substantially higher fluorescence levels, indicating that single-color screen is more prone to false positive results. On the other hand, all of the cells collected from the two-color screen maintained the low level of fluorescence after regrowing. Thirteen functional sequences were isolated from the two-color screen sort, whereas a mere 3 functional sequences were isolated from the single-color screen, with one of them being the same as one from the two-color screen. The low enrichment efficiency resulted from the single-color screen further demonstrates the resolution power of two-color screen to distinguish between functional sequences and sequences that exhibit low fluorescence levels due to gene expression noise.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of evaluating a gene-regulatory element, the method comprising:
(a) cytometrically analyzing a cell comprising a gene-regulatory construct comprising:
(i) an activity reporter comprising a first signal reporter operatively coupled to a gene-regulatory element which produces a first signal; and
(ii) a noise reporter comprising a second signal reporter which produces a second signal that is distinguishable from the first signal;
to obtain the first and second signals;
(b) normalizing the first signal with the second signal to obtain a normalized activity signal; and
(c) evaluating the gene-regulatory element from the normalized activity signal.

2. The method according to claim 1, wherein the first signal reporter encodes a first fluorescent protein and the second signal reporter encodes a second fluorescent protein.

3. The method according to claim 1, wherein the evaluating comprises determining a basal activity of the gene-regulatory element.

4. The method according to claim 1, wherein the evaluating comprises determining an activation ratio of the gene-regulatory element.

5. The method according to claim 1, wherein the gene-regulatory element is an RNA control device.

6. The method according to claim 1, wherein the gene-regulatory element is a promoter.

7. The method according to claim 1, wherein the gene-regulatory element is an RNase element.

8. The method according to claim 1, wherein the construct is a plasmid.

9. The method according to claim 1, wherein the method further comprises:
(a) cytometrically analyzing a second cell comprising a gene-regulatory construct comprising:
(i) an activity reporter comprising the first signal reporter operatively coupled to a second gene-regulatory element; and
(ii) the noise reporter;
to obtain the first and second signals;
(b) normalizing the first signal with the second signal to obtain a normalized activity signal for the second gene-regulatory element; and
(c) evaluating the second gene-regulatory element from the normalized activity signal for the second gene-regulatory element.

10. The method according to claim 9, wherein the method is method of evaluating a library of candidate gene-regulatory elements.

11. A method of evaluating a cellular library of gene-regulatory elements, the method comprising:
(a) flow cytometrically analyzing the cellular library of gene-regulatory elements comprising library members comprising a gene-regulatory construct comprising:
(i) an activity reporter comprising a first signal reporter operatively coupled to a gene-regulatory element which produces a first signal; and
(ii) a noise reporter comprising a second signal reporter which produces a second signal that is distinguishable from the first signal;
to obtain the first and second signals for library members;
(b) normalizing the first signal with the second signal to obtain a normalized activity signal for the library members; and
(c) evaluating the gene-regulatory elements in the library members from the normalized activity signal.

12. The method according to claim 11, wherein the first signal reporter encodes a first fluorescent protein and the second signal reporter encodes a second fluorescent protein.

13. The method according to claim 12, wherein the normalized activity signal is normalized fluorescent signal.

14. The method according to claim 13, wherein the evaluating comprises gating.

15. The method according to claim 14, wherein the gating is based on a normalized activity signal obtained from a control gene-regulatory element.

16. The method according to claim 11, wherein the gene-regulatory element is an RNA control device element.

17. The method according to claim 16, wherein the RNA control device element is an actuator.

18. The method according to claim 16, wherein the RNA control device element is a sensor.

19. The method according to claim 16, wherein the RNA control device element is a transmitter.

20-24. (canceled)

25. A flow cytometric system comprising:
a flow channel;
a first light source configured to direct light to an assay region of the flow channel;
a first detector configured to receive light of a first wavelength from the assay region of the flow channel;
a second detector configured to receive light of a second wavelength from the assay region of the flow channel; and
a signal processing module configured to receive signals from the first and second detectors and output a normalized activity signal to evaluate a gene-regulatory element.

26-51. (canceled)
